(19) United States
(12) Patent Application Publication
(10) Pub. No.: US 2026/0002148 A1
(43) Pub. Date: Jan. 1, 2026

(54) METHOD FOR MODULATING ACTIVITY OF CELL

(71) Applicants: UIF (University Industry Foundation), Yonsei University, Seoul (KR); INSTITUTE FOR BASIC SCIENCE, Daejeon (KR)

(72) Inventors: Jin Woo CHEON, Seoul (KR); Jae Hyun LEE, Seoul (KR); Jung Uk LEE, Seoul (KR)

(73) Assignees: UIF (University Industry Foundation), Yonsei University, Seoul (KR); INSTITUTE FOR BASIC SCIENCE, Daejeon (KR)

(21) Appl. No.: 19/321,131

(22) Filed: Sep. 5, 2025

Related U.S. Application Data
(63) Continuation-in-part of application No. 17/674,743, filed on Feb. 17, 2022.
(63) Continuation-in-part of application No. 17/674,749, filed on Feb. 17, 2022.
(63) Continuation-in-part of application No. 17/674,753, filed on Feb. 17, 2022.

(30) Foreign Application Priority Data
Feb. 17, 2021 (KR) 10-2021-0021485
Feb. 17, 2022 (KR) 10-2022-0020830
Feb. 17, 2022 (KR) 10-2022-0020901
Feb. 17, 2022 (KR) 10-2022-0020902

(51) Int. Cl. C12N 13/00; A61N 2/00; A61N 2/12

(52) U.S. Cl. CPC C12N 13/00; A61N 2/004; A61N 2/12

(57) ABSTRACT

The system for modulating the activity of cells includes a rotating magnetic field generating device which has an internal space in which a magnetic force generating unit and a living body can be disposed and forms a rotating magnetic field which satisfies Relationship Formulas 1 and 2 below; and magnetic particles disposed in the living body and capable of binding to a bioactive material and generating a torque when a rotating magnetic field is applied to transmit the torque to the bioactive material.

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

### CROSS REFERENCE TO RELATED APPLICATIONS

This application is a continuation-in-part application of U.S. application Ser. No. 17/674,743 filed Feb. 17, 2022, U.S. application Ser. No. 17/674,749 filed Feb. 17, 2022, and U.S. application Ser. No. 17/674,753 filed Feb. 17, 2022, and claims priority to and the benefit of Korean Patent Application No. 10-2021-0021485 filed Feb. 17, 2021, Korean Patent Application No. 10-2022-0020830 filed Feb. 17, 2022, Korean Patent Application No. 10-2022-0020902 filed Feb. 17, 2022; and Korean Patent Application No. 10-2022-0020901 filed Feb. 17, 2022. The contents of the above patent applications are incorporated by reference herein in their entireties for all purposes.

### GOVERNMENT SUPPORT

The present invention is derived from research conducted as part of the [IBS External Research Center] Nano-Bio Systems Convergence Science (6^{th} year) (2019-11-1707) of the Ministry of Science and ICT of the Republic of Korea (Project Identification Number: 1711122878, Detailed Task Number: IBS-R026-D1-2020-A00, Project Period: Jan. 1, 2020 to Dec. 31, 2020).

### TECHNICAL FIELD

The present invention relates to a system for modulating the activity of cells and a method for modulating the activity of cells.

### BACKGROUND ART

Various studies have been conducted to activate physiological functions or specific cells in a living body. Among these, it is classified as optogenetics, which uses a method that can activate or inactivate a specific function or cell by light at a specific wavelength. Since such optogenetics uses a non-contact method, the possibility of tissue damage is low, and the wavelength or intensity of the irradiated light can be adjusted, and thus, the degree of freedom in experimental design is high.

However, due to the low transmittance of light, the area in which the activity can be controlled is very limited, and there is a problem in that an invasive method of locating a light source into the inside of the living body must be used in order to apply the same to the inside of the living body.

### DISCLOSURE

### Technical Problem

The present invention has been devised to solve the aforementioned problems, and one of the several objects of the present invention is to provide a non-contact/non-invasive system and method for modulating the activity of cells.

One of the several objects of the present invention is to provide a system and method for modulating the activity of cells that can be applied to a wide range.

One of the several objects of the present invention is to provide a system and method for modulating the activity of cells that cannot damage a living body.

One of the several objects of the present invention is to provide a system and method for modulating the activity of cells that can modulate the activity of cells while maintaining the activity of a living body.

One of the several objects of the present invention is to provide a system and method for modulating the activity of cells cell-specifically.

### Technical Solution

The system for modulating the activity of cells according to an exemplary embodiment of the present invention may include a rotating magnetic field generating device which has an internal space in which a magnetic force generating unit and a living body can be disposed and forms a rotating magnetic field which satisfies Relationship Formulas 1 and 2 below; and magnetic particles disposed in the living body and capable of binding to a bioactive material and generating a torque when a rotating magnetic field is applied to transmit the torque to the bioactive material:

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In Relationship Formulas 1 and 2 above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

In addition, the rotating magnetic field may additionally satisfy Relationship Formula 3 below:

$$\vert {M}_{50} - {M}_{c} \vert / {D}_{50} \leq 5. T / m \qquad [ Relationship Formula 3 ]$$

In the Relationship Formula above, M_{c} is the strength of the magnetic field at the position of the rotation axis, D₅₀ is the distance from the rotation axis to the 50% position of the distance to the magnetic force generating unit, and M₅₀ is the strength of the magnetic field at the position D₅₀.

In addition, the rotating magnetic field may additionally satisfy Relationship Formula 4 below:

$$\vert {M}_{75} - {M}_{50} \vert / ( {D}_{75} - {D}_{50} ) \leq 10 T / m \qquad [ Relationship Formula 4 ]$$

In Relationship Formula 4 above, D₇₅ is the distance from the rotation axis to the 75% position of the distance to the magnetic force generating unit, D₅₀ is the distance from the rotation axis to the 50% position of the distance to the magnetic force generating unit, M₇₅ is the strength of the magnetic field at the position D₇₅, and M₅₀ is the strength of the magnetic field at the position D₅₀.

In addition, the area of the rotating magnetic field that satisfies the relationship formulas may be 0.1 cm² or more.

According to an example of the present invention, the magnetic particles may generate a torque of 10 pN·nm or more, when a rotating magnetic field that satisfies Relationship Formulas 1 and 2 above is applied.

In an exemplary embodiment of the present invention, the magnetic particles may be magnetically anisotropic (magnetic anisotropy).

In an exemplary embodiment of the present invention, the average particle diameter of the magnetic particles may be 2.0 μm or less.

Meanwhile, the magnetic particles may include a core and a plurality of nanoparticles disposed on the surface of the core.

In this case, the average particle diameter of the nanoparticles may be more than 5 nm.

In addition, the nanoparticles may include at least one selected from the group consisting of iron (Fe), manganese (Mn), nickel (Ni), zinc (Zn), aluminum (Al), cobalt (Co), chromium (Cr), molybdenum (Mo), titanium (Ti), bismuth (Bi), neodymium (Nd), platinum (Pt), gold (Au), palladium (Pd), copper (Cu), alloys thereof, oxides thereof, ferrites thereof and doped ferrites thereof.

In addition, the system may further include a linker which links the core and the nanoparticles.

In an exemplary embodiment of the present invention, the bioactive material may be a mechanosensitive channel and/or a mechanosensitive ion channel.

In this case, the mechanosensitive channel or mechanosensitive ion channel may include one or more selected from the group consisting of Piezo1, Piezo2, TRPC1, TRPC3, TRPC6, TRPM4, TRPM7, TRPN1, TRPA1, TRPY1, TRPP1, TRPP2, TRPV1, 1679K-TRPV1, TRPV2, TRPV4, TREK, TRAAK, ASIC1,2,3, MEC-4/MEC-10, MscL, MscS, RGD, integrin and cadherin.

In addition, the mechanosensitive channel and/or mechanosensitive ion channel may be opened or closed according to the application of a rotating magnetic field.

In one example, the magnetic particles of the present invention may be bound to the surface of the bioactive material.

In this case, the magnetic particles may be bound to a specific receptor or antigen located on the surface of the bioactive material.

Meanwhile, the rotating magnetic field generating device may include a plurality of magnetic field generating units.

In an exemplary embodiment of the present invention, the method for modulating the activity of cells according to the present invention may include a magnetic field application step of applying a rotating magnetic field which satisfies Relationship Formulas 1 and 2 below to magnetic particles capable of binding to a bioactive material and generating a torque when a rotating magnetic field is applied to transmit the torque to the bioactive material; and a torque transmission step of transmitting the torque generated according to the application of the rotating magnetic field to the bioactive material:

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In the relationship formulas above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

In addition, the rotating magnetic field may additionally satisfy Relationship Formula 3 below:

$$\vert {M}_{50} - {M}_{c} \vert / {D}_{50} \leq 1 T / m \qquad [ Relationship Formula 3 ]$$

In Relationship Formula 3 above, M_{c} is the strength of the magnetic field at the position of the rotation axis, D₅₀ is the distance from the rotation axis to the 50% position of the distance to the magnetic force generating unit, and M₅₀ is the strength of the magnetic field at the position D₅₀.

In addition, the rotating magnetic field may additionally satisfy Relationship Formula 4 below:

$$\vert {M}_{75} - {M}_{50} \vert / ( {D}_{75} - {D}_{50} ) \leq 10 T / m \qquad [ Relationship Formula 4 ]$$

In Relationship Formula 4 above, D₇₅ is the distance from the rotation axis to the 75% position of the distance to the magnetic force generating unit, D₅₀ is the distance from the rotation axis to the 50% position of the distance to the magnetic force generating unit, M₇₅ is the strength of the magnetic field at the position D₇₅, and M₅₀ is the strength of the magnetic field at the position D₅₀.

In this case, the magnetic particles may generate a torque of 10 pN·nm or more, when a rotating magnetic field that satisfies Relationship Formulas 1 and 2 above is applied.

In an exemplary embodiment of the present invention, the torque transmission step may be a step of transmitting a torque generated by the magnetic particles through the surface of the bioactive material to which the magnetic particles are bound.

In addition, the bioactive material may be a mechanosensitive channel and/or a mechanosensitive ion channel.

In this case, the mechanosensitive channel and/or mechanosensitive ion channel may be opened or closed according to the application of a rotating magnetic field.

In one example of the present invention, the method may further include a magnetic particle attachment step performed before the magnetic field application step, wherein the magnetic field attachment step may be a step of binding magnetic particles to a specific receptor or antigen on a bioactive material.

In this case, the present invention may further include a step of activating cells using a torque transmitted to the bioactive material.

Meanwhile the activated cells may include nerve cells, glial cells, immune cells and/or cancer cells.

### Advantageous Effects

One of the several effects of the present invention is that it is possible to modulate the activity of cells without contacting the cells or living body.

One of the several effects of the present invention is that it is possible to provide a system and method for modulating the activity of cells that can avoid using invasive methods.

One of the several effects of the present invention is that it is possible to modulate the activity of cells in a wide range.

One of the several effects of the present invention is that it is possible to provide a system and method for modulating the activity of cells that cannot damage the living body.

One of the several effects of the present invention is that it is possible to provide a system and method for modulating the activity of cells that can modulate the activity of cells while maintaining the activity of the living body.

One of the several objects of the present invention is to provide a system and method for modulating the activity of cells cell-specifically.

### DESCRIPTION OF DRAWINGS

The patent or application file contains at least one drawing executed in color. Copies of this patent or patent application publication with color drawings will be provided by the Office upon request and payment of the necessary fee.

FIG. 1 is a perspective view schematically illustrating the rotating magnetic field generating device for modulating the activity of cells according to an exemplary embodiment of the present invention.

FIG. 2 is a plan view of FIG. 1.

FIG. 3 is a perspective view illustrating a case in which there were six magnetic force generating units of the rotating magnetic field generating device of FIG. 1.

FIG. 4 is a plan view of FIG. 3.

FIG. 5 is a first modification example of FIG. 1.

FIG. 6 is a second modification example of FIG. 1.

FIG. 7 is a perspective view schematically illustrating the rotating magnetic field generating device for modulating the activity of cells according to another exemplary embodiment of the present invention.

FIG. 8 is a side view of FIG. 7.

FIG. 9 schematically depicts the direction of magnetic force lines in the structure of FIG. 8.

FIG. 10 is a first modification example of FIG. 7.

FIG. 11 is a side view of FIG. 10.

FIG. 12 is a second modification example of FIG. 7.

FIG. 13 is a mimetic diagram schematically illustrating the structure of magnetic particles applied to the rotating magnetic field generating device for modulating the activity of cells according to the present invention.

FIG. 14 is a set of TEM images obtained by photographing nanoparticles attached to the surface of the magnetic particles according to the present invention.

FIG. 15 is a set of TEM images obtained by photographing magnetic particles having nanoparticles attached to the surface of the core according to an exemplary embodiment of the present invention.

FIG. 16 is an image showing a simulation result of the residual magnetization of nanoparticles at room temperature using the micro-magnetic simulation program (OOMMF) and a graph of the residual magnetization.

FIG. 17 is an image visualized by mathematically calculating the strength of a magnetic field according to changes in the number of magnetic force generating units and the longest distance between a plurality of magnetic force generating units.

FIG. 18 is a set of images visualized by mathematically calculating the strength of magnetic fields according to the distance from the rotation axis respectively for a case where there were 6 magnetic force generating units and the longest distance between the plurality of magnetic force generating units was 16 cm, and a case where there were 10 magnetic force generating units and the longest distance between the magnetic force generating units was 70 cm.

FIG. 19 is a graph showing the relationship between the size of magnetic particles and the number of magnetic particles bound to cells.

FIG. 20 is a set of TEM images obtained by photographing the movement of magnetic particles when the gradient of the magnetic field exceeded Relationship Formula 3 according to the present invention.

FIG. 21 is a set of images obtained by photographing with a fluorescence microscope the states in which a fluorescent material was attached to one part of the magnetic particles according to Example 1 of the present invention and a predetermined rotating magnetic field was applied.

FIG. 22 is a set of images obtained by photographing with a fluorescence microscope the states in which a fluorescent material was attached to one part of the magnetic particles according to Comparative Example 1 of the present invention and a predetermined rotating magnetic field was applied.

FIG. 23 is a set of immune-stained fluorescence images obtained by photographing a fluorescent material stained using the Myc antibody after expressing the Piezo1 channel in cortical neurons of mice.

FIG. 24 is a set of images obtained photographing with a fluorescence microscope the distribution of magnetic particles in the cortical neurons of mice of FIG. 23.

FIG. 25 is a set of images obtained by photographing Ca²⁺ ion-sensitized fluorescence signals (X-Rhod-1) when the neurons of mice in FIG. 23 were placed in the rotating magnetic field generating device according to the present invention and then a rotating magnetic field was applied at a rate of 0.5 Hz.

FIG. 26 is a set of images obtained by photographing Ca²⁺ ion-sensitized fluorescence signals (X-Rhod-1) when no rotating magnetic field was applied to the neurons of mice in FIG. 23.

FIG. 27 is a set of images obtained by photographing Ca²⁺ ion-sensitive fluorescence signals (X-Rhod-1) when a rotating magnetic field was applied after binding magnetic particles to WGA, which non-specifically binds to cortical neurons of mice.

FIG. 28 is a set of images obtained by photographing Ca²⁺ ion-sensitive fluorescence signals (X-Rhod-1) when a rotating magnetic field was applied after additional injection of Ruthenium Red, which inactivates the Piezo1 channel, into the neurons of mice in FIG. 23.

FIG. 29 is a graph showing the results of measuring the force generated by changing the strength of the magnetic field with respect to the magnetic particles of Example 3 and Comparative Examples 4 and 5.

### MODES OF THE INVENTION

Hereinafter, exemplary embodiments of the present invention will be described with reference to specific examples and the accompanying drawings. It is not intended to limit the technology described herein to specific exemplary embodiments, and it is to be understood that various modifications, equivalents and/or alternatives of the exemplary embodiments of the present invention are included. In connection with the description of the drawings, like reference numerals may be used for like components.

In addition, in order to clearly describe the present invention in the drawings, parts that are irrelevant to the description are omitted, and the thickness is enlarged to clearly express various layers and regions, and components having the same function within the scope of the same spirit may be described by using the same reference numerals.

In the present specification, expressions such as “have”, “may have”, “include” or “may include” indicate the presence of a corresponding feature (e.g., a numerical value, function, operation or component such as a part). and do not exclude the presence of additional features.

In the present specification, expressions such as “A or B”, “at least one of A and/or B” or “one or more of A or/and B” may include all possible combinations of the items listed together. For example, “A or B”, “at least one of A and B” or “at least one of A or B” may refer to all cases of (1) including at least one A, (2) including at least one B, or (3) including at least one A and at least one B.

The present invention relates to a system for modulating the activity of cells. The system for modulating the activity of cells according to the present invention may include a rotating magnetic field generating device which has an internal space in which a magnetic force generating unit and a living body can be disposed and forms a rotating magnetic field; and magnetic particles disposed in the living body and capable of binding to a bioactive material and generating a torque when a rotating magnetic field is applied to transmit the torque to the bioactive material:

In this case, the rotating magnetic field may satisfy Relationship Formulas 1 and 2 below.

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In Relationship Formulas 1 and 2 above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

Hereinafter, each configuration of the system for modulating the activity of cells will be described separately.

### Rotating Magnetic Field Generating Device

Recent research on biological systems is progressing in various directions, and various methods for activating physiological functions or specific cells in the living body have been suggested. Optogenetics is a representative of these methods, and it has been reported that a predetermined function or cell can be activated by irradiating light at a specific wavelength to an optogenetic actuator such as channelrhodopsin.

However, unlike the point initially suggested as an advantage of optogenetics in the use of a non-contact method, the limitation of using the invasive method in the biological unit rather than the cell unit has been revealed. This is because light near the wavelength range of visible light has low penetrating power to the living body, and in order to control the activity by irradiating light to cells or organs inside the living body, there is a problem in that a light source must be located inside the living body through a method such as surgery.

On the other hand, the magnetic field has the advantage of having a high penetrating power with respect to the living body. By using this, magnetogenetics, which activates or inactivates specific functions or cells of a living body, is being studied as a new method. In the case of using a magnetic field, it is possible to apply a sufficient magnetic field even outside the living body such that irreversible damage to the living body can be fundamentally prevented.

However, the strength of the magnetic field can be explained by the magnetic flux density (B), and since the magnetic flux density is inversely proportional to the distance from the source, it is very difficult to apply a magnetic field having a certain strength to a long distance. For this reason, existing studies on magnetic fields have only implemented cell activity at short distances. Accordingly, the inventors of the present invention have discovered that the activity of a target cell in a living body can be regulated by using a rotating magnetic field and magnetic particles that satisfy the above-described relationship formulas. The present invention has been devised from the above studies, and the system and method for modulating the activity of cells according to the present invention can control the activity of a target cell and a living body including the same while using a non-contact/non-invasive method.

Accordingly, the inventors of the present invention have discovered that the activity of a target cell can be modulated in vivo by applying a rotating magnetic field in a predetermined range. The present invention has been devised from the above studies, and the magnetic particle for modulating the activity of cells according to the present invention can generate an appropriate level of torque while minimizing linear motion when a rotating magnetic field is applied, which will be described below. Through this, the magnetic particle for modulating the activity of cells according to the present invention can modulate the activity of a target cell and a living body including the same through a non-contact/non-invasive method.

The system for modulating the activity of cells according to the present invention may include a rotating magnetic field generating device which has an internal space in which a magnetic force generating unit and a living body can be disposed and forms a rotating magnetic field. As used herein, the term “rotating magnetic field” may mean a magnetic field in which lines of magnetic force are rotated in a direction perpendicular to the rotation axis around a virtual rotation axis, and it may refer to a magnetic field in which the direction of the magnetic field appears to rotate with a predetermined angular velocity over time. For example, the angular velocity of the rotating magnetic field may be 0.01 Hz or more and/or 10,000 Hz or less, but is not limited thereto. In addition, in the present specification, an imaginary plane in which the magnetic flux direction of the rotating magnetic field is horizontal is defined as a “reference plane”, and the “strength of magnetic field” may mean a value measured on the reference plane. In the present specification, the “distance from the rotation axis to the magnetic force generating unit” may mean an arithmetic average of distances from the rotation axis to a plurality of magnetic force generating units. In addition, although the reference plane is depicted in a disk shape in the present specification and drawings, this is a description for easily grasping the position of the reference plane, and the reference plane in the drawing may not be a tangible entity.

In this case, the rotating magnetic field may satisfy Relationship Formula 1 and 2 below.

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In Relationship Formulas 1 and 2 above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

Relationship Formula 1 relates to the strength of the magnetic field at the position of the rotation axis. The system for modulating the activity of cells according to the present invention generates a torque using a rotating magnetic field, and as the direction of magnetic flux rotates, the torque can be transmitted to the bioactive material through magnetic particles bound to the bioactive material. In this case, the strength of the magnetic field becomes weaker as it moves away from the magnetic force generating unit, but when Relationship Formula 1 above is satisfied, a torque having at least a certain level can be generated even in the central part of the rotating magnetic field generating device, and through this, it is possible to modulate the activity of cells.

Relationship Formula 2 shows the relationship between the strength of the magnetic field at the position of the rotation axis and the strength of the magnetic force at the 75% position of the distance from the rotation axis to the magnetic force generating unit. Looking at Relationship Formula 2 above, the rotational magnetic field satisfying Relationship Formula 2 has a change rate of the strengths of the magnetic field up to the 75% position of the distance from the rotation axis to the magnetic force generating unit, that is, the gradient of the strength of the magnetic field according to the distance from the rotation axis may satisfy 5.0 T/m or less. This may mean that the change value of the strengths of the rotating magnetic field formed by the rotating magnetic field generating device of the system for modulating the activity of cells according to the present invention is not large. When Relationship Formula 2 above is satisfied, it is possible to form a uniform rotating magnetic field in the region up to a predetermined distance from the central part, thereby maximizing the internal space of the rotating magnetic field generating device, and through this, it is possible to modulate the activity of cells over a wide area.

In one example of the present invention, the rotating magnetic field formed by the rotating magnetic field generating device of the present invention may further satisfy Relationship Formula 3 below.

$$\vert {M}_{50} - {M}_{c} \vert / {D}_{50} \leq 1 T / m \qquad [ Relationship Formula 3 ]$$

In Relationship Formula 3 above, M_{c} is the strength of the magnetic field at the position of the rotation axis, D₅₀ is the distance from the rotation axis to the 50% position of the distance to the magnetic force generating unit, and M₅₀ is the strength of the magnetic field at the position D₅₀.

Relationship Formula 3 relates to the strength of the magnetic field at the position of the rotation axis and the strength of the magnetic field in the middle region between the position of the rotation axis and the magnetic force generating unit. In the rotating magnetic field satisfying Relationship Formula 3 above, the slope of the strength of the magnetic field according to the distance from the rotation axis may satisfy 1.0 T/m or less. When Relationship Formula 3 above is satisfied, the strength of the magnetic field may be substantially constant in the central region around the rotation axis. The system for modulating the activity of cells according to the present invention can apply a uniform rotating magnetic field with a very small change in the strength of the magnetic field to a region having a certain area or more of the internal space by forming a rotating magnetic field that satisfies Relationship Formula 3 above, and through this, it may be possible to precisely modulate the activity of cells over a wide area.

In one example of the present invention, the rotating magnetic field formed by the rotating magnetic field generating device of the present invention may further satisfy Relationship Formula 4 below.

$$\vert {M}_{75} - {M}_{50} \vert / ( {D}_{75} - {D}_{50} ) \leq 10 T / m \qquad [ Relationship Formula 4 ]$$

In Relationship Formula 4 above, D₇₅ is the distance from the rotation axis to the 75% position of the distance to the magnetic force generating unit, D₅₀ is the distance from the rotation axis to the 50% position of the distance to the magnetic force generating unit, M₇₅ is the strength of the magnetic field at the position D₇₅, and M₅₀ is the strength of the magnetic field at the position D₅₀.

Relationship Formula 4 relates to the strength of the magnetic force at the 75% position of the distance from the rotation axis to the magnetic force generating unit, and the strength of the magnetic force at the 50% position of the distance from the rotation axis to the magnetic force generating unit. If aforementioned Relationship Formulas 2 and 3 relate to the change rate of the strengths of the magnetic field in the central portion of the rotating magnetic field generating device, Relationship Formula 4 above represents the change rate of the strengths of the magnetic field in the outer portion of the rotating magnetic field generating device. When Relationship Formula 3 above is satisfied, the change rate of the strengths of the magnetic field in the outer portion of the internal space of the rotating magnetic field generating device may be suppressed within a predetermined range, and it is possible to modulate the activity of cells in a wider space.

In one example, the rotating magnetic field formed by the rotating magnetic field generating device for modulating the activity of cells according to the present invention may satisfy Relationship Formula 1 described above and may satisfy at least one or more of Relationship Formulas 2 to 4 at the same time. When the rotating magnetic field of the rotating magnetic field generating device according to the present invention satisfies Relationship Formula 1 and satisfies at least one or more of Relationship Formulas 2 to 4 at the same time, the variation in the strength of the magnetic field applied to the internal space of the rotating magnetic field generating device may be reduced, and thus, it is possible to apply a magnetic field having constant strength to a large area.

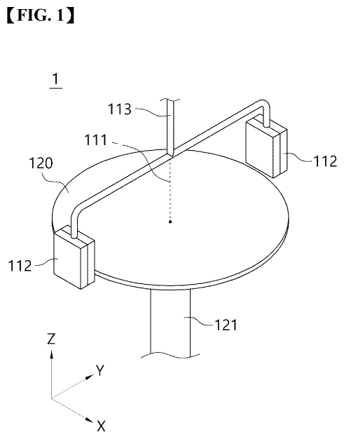

FIG. 1 is a perspective diagram schematically illustrating the rotating magnetic field generating device of the present invention. Referring to FIG. 1, the rotating magnetic field generating device of the system and method for modulating the activity of cells according to the present invention forms a rotating magnetic field with a virtual reference line as a rotation axis 111, and may include an internal space in which a plurality of magnetic force generating units 112 that are spaced apart from the rotation axis 111; and a living body can be disposed, and the reference plane 120 on which the magnetic force lines of the rotating magnetic field are horizontal may be located in the internal space.

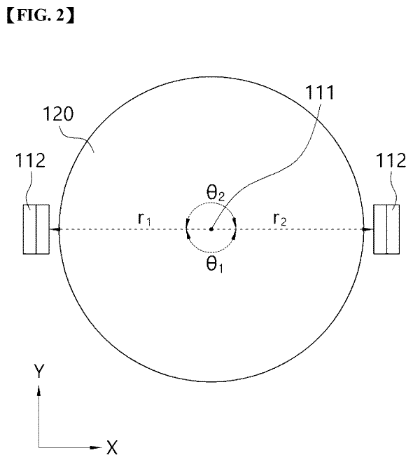

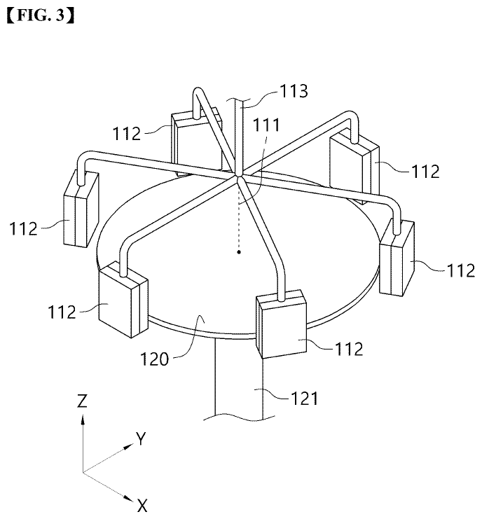

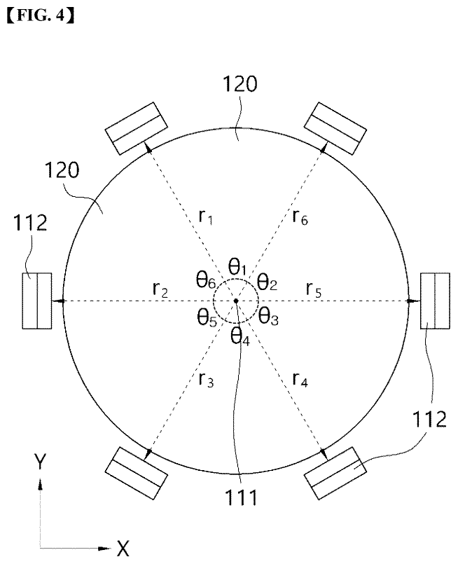

The rotating magnetic field generating device according to the present invention may include a plurality of magnetic force generating units 112. For example, the number of the magnetic force generating units 112 may be 2 or more, 3 or more, 4 or more, 5 or more or 6 or more, but is not limited thereto. However, the magnetic force generating unit 112 must be arranged to form a rotating magnetic field, and specifically, the magnetic force generating unit 112 must be arranged such that the magnetic force line indicating the direction of the magnetic field passes on the rotation axis 111. FIGS. 1 and 2 are mimetic diagrams schematically illustrating structures in which there are two magnetic force generating units 112, and FIGS. 3 and 4 are mimetic diagrams schematically illustrating structures in which there are six magnetic force generating units 112. As illustrated in FIGS. 1 to 4, the rotating magnetic field generating device 1 according to the present invention may include a plurality of magnetic force generating units 112 if the rotating magnetic field can be formed. In addition, the upper limit of the number of the magnetic force generating units 112 is not particularly limited as long as it can form a rotating magnetic field that satisfies the above-described relationship formulas, and it may be, for example, 50 or less.

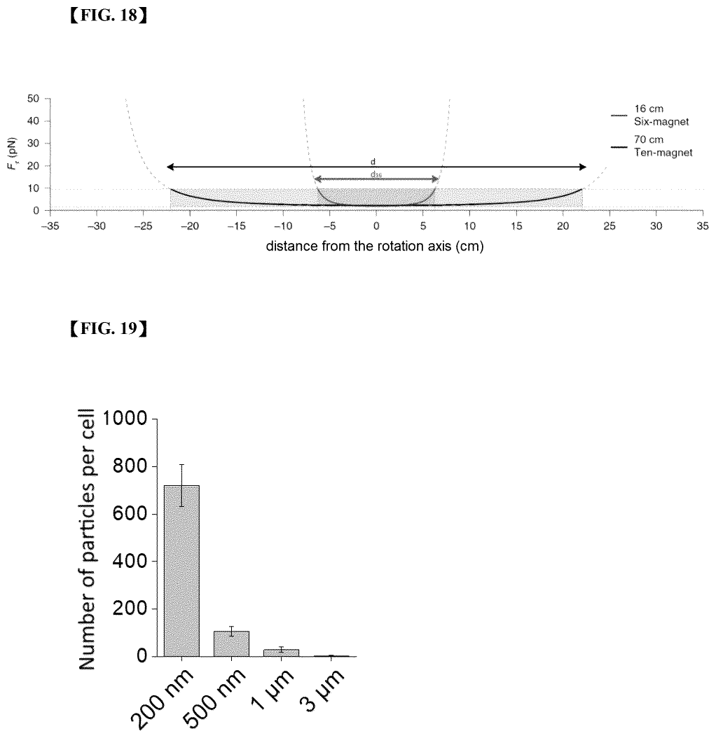

FIG. 17 is a visualization of mathematical calculations of the strength of a magnetic field according to changes in the number of magnetic force generating units and the longest distance between a plurality of magnetic force generating units (hereinafter, referred to as the “long axis” of the rotating magnetic field generating device). Referring to FIG. 17, it can be confirmed that it was very difficult to form a magnetic field with uniform strength when the number of magnets of the rotating magnetic field generating device was two and the length of the long axis was 8 cm, and when the number of magnets was two and the length of the long axis was 12 cm, it can be confirmed that a region in which the strength of the magnetic field was maintained constant was formed to some extent. On the other hand, when the number of magnets was 4 or 6 and the length of the long axis was 16 cm, it can be seen that a magnetic field with uniform strength was formed in a very wide area. In addition, referring to FIG. 18, when the number of magnets was 10 and the length of the long axis was 70 cm, it can be seen that a uniform rotating magnetic field could be formed in a very wide range, compared to the case where 6 magnets were used and the length of the long axis was 16 cm. The rotating magnetic field generating device according to the present invention can modulate the activity of cells over a wide area by forming a uniform rotating magnetic field that does not have a large change in strength inside the device as described above. Detailed experimental data will be described below through the examples.

The type of the magnetic force generating unit 112 is not particularly limited as long as it can generate a sufficient level of magnetic force. For example, a permanent magnet such as neodymium or an electromagnet using a coil or the like may be used. In addition, although FIGS. 1 to 4 illustrate configurations in which the magnet rotates about the rotation axis 111, the structure is not particularly limited as long as the magnetic flux direction can be constantly rotated. For example, by adjusting the phase of a sine wave in an AC motor having a plurality of axes to rotate the direction of magnetic flux, it may have a structure in which the direction of the magnetic field is rotated while having a fixed magnetic field generating unit.

In an exemplary embodiment of the present invention, the area of the rotating magnetic field satisfying at least one of Relationship Formulas 2 to 4 while satisfying above-described Relationship Formula 1 may be 0.1 cm² or more. Conventional rotating magnetic field generating devices have a limitation in that the strength of the magnetic field according to the location is significantly different even if it forms a rotating magnetic field over a wide area applicable to the DNA level or a rotating magnetic field over a wide area. Due to this, even if a rotating magnetic field is used, it can be applied to a very narrow area or can be applied only to a field that is less affected even if the strength of the magnetic field is greatly changed. As described above, the rotating magnetic field generating device of the system for modulating the activity of cells according to the present invention has a plurality of magnetic force generating units, and can form a uniform rotating magnetic field over a large area by adjusting the distance between the plurality of magnetic force generating units.

The area of the rotating magnetic field satisfying at least one of Relationship Formulas 2 to 4 while satisfying above-mentioned Relationship Formula 1 may be 0.1 cm² or more. 0.2 cm² or more, 0.3 cm² or more, 0.4 cm² or more, 0.5 cm² or more, 0.6 cm² or more, 0.7 cm² or more, 0.8 cm² or more, 0.9 cm² or more or 1.0 cm² or more, but is not limited thereto. In addition, the upper limit of the area of the rotating magnetic field satisfying the above relationship formulas is not particularly limited, but may be, for example, 10 m² or less. Since the rotating magnetic field generating device of the present invention can form a uniform rotating magnetic field over a large area, even when a target to activate cells by applying a rotating magnetic field is a living body, it is possible to modulate the activity of cells while maintaining the activity of the living body.

In one example, the area on the reference plane that satisfies the above-described relationship formulas among the rotating magnetic fields applied in the rotating magnetic field generating device for modulating the activity of cells according to the present invention may be 50% or more of the total area of the reference plane. The rotating magnetic field generating device illustrated in FIGS. 1 to 6 exemplifies a case in which a plurality of magnetic force generating units 112 have a structure disposed very close to the reference plane 120 of the internal space. In this case, the area of the rotating magnetic field on the reference plane 120 that satisfies the above-described relationship formulas with respect to the central rotation axis 111 may be the remaining region except for the vicinity of the magnetic force generating unit 112. For example, referring to FIGS. 2 and 4, the magnetic force generating unit 112 may be disposed to be spaced apart from the rotation axis 111, and the horizontal area of the space within the separation distance may mean the total area. The distance at which the magnetic force generating unit 112 is separated from the rotation axis 111 may be the same, but may not necessarily be the same. In this case, when the separation distance between the magnetic force generating unit 112 and the rotation axis 111 is not the same, the internal space may be calculated based on the minimum value among the separation distances. In the case of FIG. 2, the horizontal area of the internal space can be calculated based on the smaller value of r1 and r2, and in the case of FIG. 4, the horizontal area of the internal space may be calculated based on a minimum value among r1, r2, r3, r3, r4, r5 and r6.

The upper limit of the area of the rotating magnetic field on the reference plane 120 satisfying the above relationship formulas is not particularly limited, but may be, for example, 100%. Unlike the above structure, when the internal space is spaced apart from the magnetic force generating unit 112 in the XY direction, that is, when the internal space is formed narrower than the distance between the magnetic force generating units 112, all of the rotating magnetic fields formed on the reference plane 120 of the internal space may satisfy the above-described relationship formulas.

In an exemplary embodiment of the present invention, the rotating magnetic field generating device for modulating the activity of cells according to the present invention may further include a support 121 connected to the internal space and a driving unit 113 connected to the magnetic force generating unit 112 as needed. When a container capable of locating a living body is disposed in the internal space of the rotating magnetic field generating device for modulating the activity of cells according to the present invention, the support 121 may be selectively disposed to serve to support the container. In addition, when the magnetic force generating unit is a permanent magnet, for example, the driving unit 113 must physically move the magnetic force generating unit to generate a rotating magnetic field, and in this case, it may be selectively disposed to fix and rotate the magnetic force generating unit.

In one example of the above exemplary embodiment, the support may be disposed in the height direction of the internal space, and the driving unit may be disposed in a direction opposite to the direction in which the support is disposed. Referring to FIGS. 1 and 3, the support 121 of the rotating magnetic field generating device for modulating the activity of cells according to the present invention may be disposed downward in the height direction (Z direction), and the driving unit 113 may be disposed upward in the height direction (Z direction), which is the opposite direction in which the support 121 is disposed.

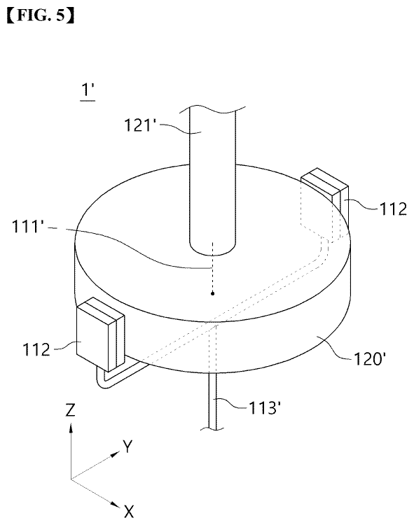

In addition, FIG. 5 schematically illustrates a form opposite to the structures of FIGS. 1 and 3. Referring to FIG. 5, the support 121′ of the rotating magnetic field generating device for modulating the activity of cells according to the present invention may be disposed upward in the height direction (Z direction), and the driving unit 113′ may be disposed downward in the height direction (Z direction) opposite to the direction of the support 121′.

That is, the structure of this example may be a structure in which the reference plane 120 is disposed between the supports 121, 121′ and the driving units 113, 113′. As in this example, when the supports 121, 121′ and the driving units 113, 113′ are disposed to face each other in opposite directions, the structure of the rotating magnetic field generating device for modulating the activity of cells may be formed simply, and it may be easy to change the structure according to the purpose of application.

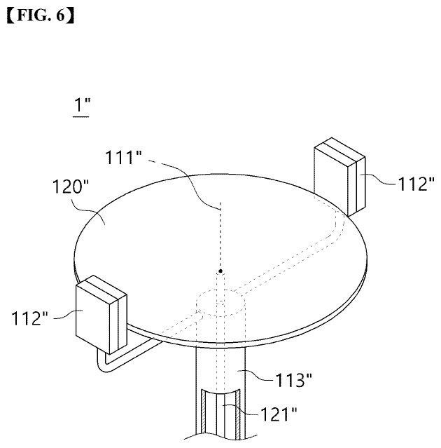

In another example of the above exemplary embodiment, the support and the driving unit may be disposed in the height direction of the internal space, and the support and the driving unit may be disposed in the same direction. FIG. 6 schematically illustrates the rotating magnetic field generating device for modulating the activity of cells according to this example. Referring to FIG. 6, both of the support 121″ and the driving unit 113″ may be disposed downward in the height direction (Z direction). In this case, the driving unit 113″ may have a structure separate from the support 121″, and may have a structure that rotates independently. As in this example, when the support 121″ and the driving unit 113″ are disposed in the same direction in the height direction (Z direction), there is no structure located on the upper part of the reference plane, and thus, it is possible to freely utilize the upper space of the reference plane, and it is possible to modulate the activity of cells for a sample having a large size.

The rotating magnetic field generating device for modulating the activity of cells according to another exemplary embodiment of the present invention may include an internal space in which a plurality of magnetic force generating units which form a rotating magnetic field with a virtual reference line as a rotation axis, and are spaced apart from the rotation axis; and a living body can be disposed. In this case, the rotating magnetic field on the reference plane may satisfy Relationship Formulas 1 and 2 below.

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In Relationship Formulas 1 and 2 above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

Since the descriptions for Relationship Formulas 1 and 2 above are the same as those described above, they will be omitted.

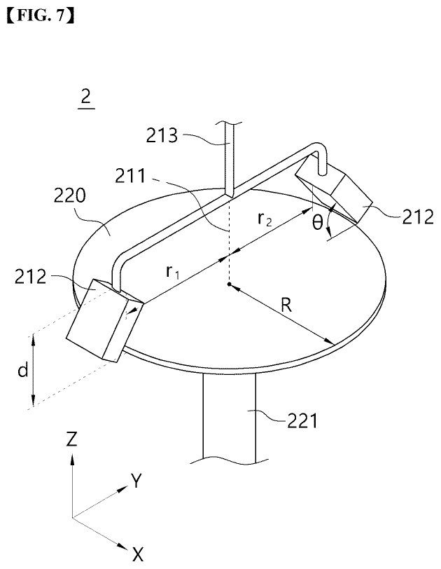

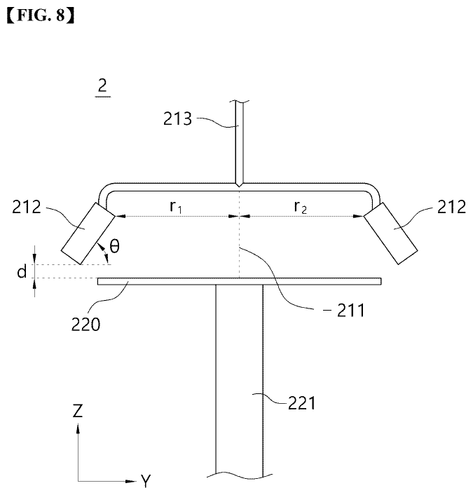

FIGS. 7 and 8 are perspective views schematically illustrating the rotating magnetic field generating device 2 for modulating the activity of cells according to the present exemplary embodiment. Referring to FIGS. 7 and 8, the rotating magnetic field generating device 2 according to the present exemplary embodiment may include a plurality of magnetic force generating units 212 arranged to be spaced apart from the reference plane 220 by a predetermined distance (d) in the height direction.

The magnetic force line indicating the direction of the magnetic field is displayed as a continuous line from one pole to the other pole, and in this case, the magnetic force line does not break or intersect between the two poles, and the direction of the magnetic force line at positions spaced apart by a certain distance respectively from the two poles may have the same direction as the straight line between the two poles. The rotating magnetic field generating device 2 according to the present exemplary embodiment may be disposed to be spaced apart by a predetermined distance (d) in the height direction from the reference plane 220 to form a magnetic force line in a parallel direction on the reference plane 220, and may form a rotating magnetic field according to the rotation of the magnetic force line. Since the reference plane 220 is disposed to be spaced apart from both poles respectively by a certain distance or more, the uniformity of the strength of the magnetic field on the reference plane 220 may be further improved. In addition, the magnetic force generating unit 212 may form a large space above the reference plane 220 by a distance (d) that is spaced apart from the reference plane 220 so as to apply a rotating magnetic field over a wider area.

In one example, the magnetic force generating unit 212 of the rotating magnetic field generating device 2 for modulating the activity of cells may be disposed to form an angle of less than 90° with respect to the reference plane. Referring to FIG. 8, it can be confirmed that the magnetic force generating unit 212 and the reference plane 220 form a predetermined angle (θᵥ). As such, when the magnetic force generating unit 212 is disposed to form an angle of less than 90° with the reference plane 220, the direction of the magnetic force line may be more easily adjusted, and the distance between the magnetic force generating unit 212 and the reference plane 220 of the internal space may become longer.

The lower limit of the angle (θᵥ) between the magnetic force generating unit 212 and the reference plane 220 is not particularly limited, but may be, for example, 0° or more or more than 0°. The magnetic force generating unit may include an adjusting unit (not illustrated) capable of adjusting the angle formed with the reference plane. For example, when the magnetic force generating unit is connected to the driving shaft, the adjusting unit may be disposed between the driving shaft and the magnetic force generating unit so as to adjust the angle between the magnetic force generating unit and the reference plane.

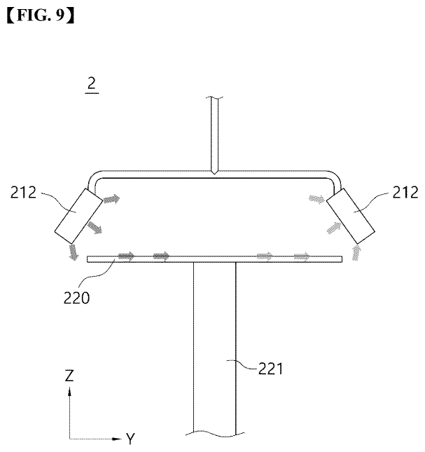

FIG. 9 is a mimetic diagram schematically illustrating the magnetic force lines of the above example. In FIG. 9, the arrow indicates the direction of the magnetic force line. Referring to FIG. 9, when the magnetic field generating unit 212 has a predetermined angle with respect to the reference plane 220, the direction of the magnetic force lines emitted from the magnetic force generating unit 212 on one side may not be a horizontal direction. Specifically, the direction of the emitted magnetic force line may form a constant angle (90°−θ) with the reference plane. The magnetic force line emitted to form a constant angle (90°−θ) with the reference plane leads to the magnetic force generating unit 212 on the other side, and also forms a predetermined angle with the magnetic field generating unit 212 on the other side. In addition, the reference plane 220 in which the direction of the magnetic field is in a horizontal direction may be located at a position spaced apart by a predetermined distance (d) from the magnetic force generating units 212 on both sides.

In the present exemplary embodiment, as the magnetic field generating unit is arranged to have a predetermined angle with respect to the reference plane as described above, the position at which the direction of the magnetic field is horizontal may be adjusted to be formed to be spaced apart from the magnetic force generating unit by a predetermined distance, and it is possible to form a reference plane at a position spaced apart from the magnetic force generating unit by a certain distance. Through this, it is possible to form a larger range in which the strength of the magnetic field is uniform, and it is possible to maximize the internal space of the rotating magnetic field generating device for modulating the activity of cells according to the present invention.

In addition, the rotating magnetic field applied by the rotating magnetic field generating device for modulating the activity of cells according to the present exemplary embodiment may satisfy Relationship Formula 3 below.

$$\vert {M}_{50} - {M}_{c} \vert / {D}_{50} \leq 1 T / m \qquad [ Relationship Formula 3 ]$$

In Relationship Formula 3 above, M_{c} is the strength of the magnetic field at the position of the rotation axis, D₅₀ is the distance from the rotation axis to the 50% position of the distance to the magnetic force generating unit, and M₅₀ is the strength of the magnetic field at the position D₅₀.

Meanwhile, the rotating magnetic field applied by the rotating magnetic field generating device for modulating the activity of cells according to the present exemplary embodiment may satisfy Relationship Formula 4 below.

$$\vert {M}_{75} - {M}_{50} \vert / ( {D}_{75} - {D}_{50} ) \leq 10 T / m \qquad [ Relationship Formula 4 ]$$

In Relationship Formula 4 above, D₇₅ is the distance from the rotation axis to the 75% position of the distance to the magnetic force generating unit, D₅₀ is the distance from the rotation axis to the 50% position of the distance to the magnetic force generating unit, M₇₅ is the strength of the magnetic field at the position D₇₅, and M₅₀ is the strength of the magnetic field at the position D₅₀.

Since the descriptions of Relationship Formulas 3 and 4 are the same as those described above, they will be omitted.

The rotating magnetic field formed by the rotating magnetic field generating device for modulating the activity of cells according to the present exemplary embodiment may satisfy Relationship Formula 1 described above and may satisfy at least one or more of Relationship Formulas 2 to 4 at the same time. Since the descriptions of Relationship Formulas 3 and 4 are the same as those described above, they will be omitted.

The rotating magnetic field generating device may include a plurality of magnetic force generating units 212, and for example, the number of magnetic force generating units 212 may be 2 or more, 3 or more, 4 or more, 5 or more or 6 or more, and may be 50 or less, but is not limited thereto.

In an exemplary embodiment of the present invention, the area of the rotating magnetic field that satisfies aforementioned Relationship Formula 1 and at least one or more of Relationship Formulas 2 to 4 at the same time may be 0.1 cm² or more, and the upper limit is not particularly limited, but may be, for example, 10 m² or less.

In one example, the area on the reference plane that satisfies aforementioned Relationship Formula 1 and at least one or more of Relationship Formulas 2 to 4 among the rotating magnetic fields applied by the rotating magnetic field generating device for modulating the activity of cells according to the present invention may be 50% or more of the total area of the reference plane. The rotating magnetic field generating device shown in FIGS. 7 to 12 exemplifies a case in which a plurality of magnetic force generating units 212 have a structure disposed very close to the reference plane 220 of the internal space. In this case, the area of the rotating magnetic field on the reference plane 220 that satisfies the above-described relationship formulas with respect to the central rotation axis 211 may be the remaining area except for the vicinity of the magnetic force generating unit 212. The upper limit of the area of the rotating magnetic field on the reference plane 220 satisfying the above relationship formulas is not particularly limited, but may be, for example, 100%. Unlike the above structure, when the internal space is disposed to be spaced apart from the magnetic force generating unit 212 in the XY direction, the rotating magnetic field formed on the reference plane 220 of the internal space may all satisfy the above-described relationship formulas.

In an exemplary embodiment of the present invention, the rotating magnetic field generating device for modulating the activity of cells according to the present invention may further include a support connected to the internal space and a driving unit connected to the magnetic force generating unit as needed.

In one example of the above exemplary embodiment, the support may be disposed in the height direction of the internal space, and the driving unit may be disposed in a direction opposite to the direction in which the support is disposed. Referring to FIGS. 7 and 8, the support 221 of the rotating magnetic field generating device 2 for modulating the activity of cells according to the present invention may be disposed downward in the height direction (Z direction), and the driving unit 213 may be disposed to face upward in the height direction (Z direction), which is the opposite direction in which the support 221 is disposed.

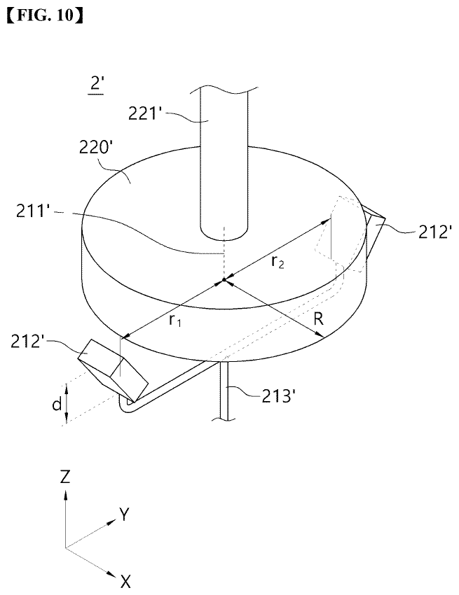

In addition, FIGS. 10 and 11 schematically illustrate structures opposite to those of FIGS. 7 and 8. Referring to FIGS. 10 and 11, the support 221′ of the rotating magnetic field generating device for modulating the activity of cells according to the present invention may be disposed upward in the height direction (Z direction), and the driving unit 213′ may be disposed downward in the height direction (Z direction), which is the opposite direction to the support 221′.

In another example of the above exemplary embodiment, the support and the driving unit may be disposed in the height direction of the internal space, and the support and the driving unit may be disposed in the same direction. FIG. 12 schematically illustrates the rotating magnetic field generating device for modulating the activity of cells according to this example. Referring to FIG. 12, both the support 221″ and the driving unit 213″ may be disposed to face downward in the height direction (Z direction). In this case, the driving unit 213″ may have a structure separate from the support 221″, and may have a structure that rotates independently.

Since the descriptions of the support and the driving unit are the same as those described above, they will be omitted.

In one example, a plurality of magnetic field generating units included in the rotating magnetic field generating device for modulating the activity of cells according to the present invention may be arranged at a predetermined angle about the rotation axis. In this case, the angles of at least two or more magnetic force generating units among the angles formed by the plurality of magnetic field generating units may be arranged to be different from each other. For example, referring to FIG. 2, when there are two magnetic force generating units, angles (θ1, θ2) between the two magnetic force generating units about the rotation axis may be different from each other. In addition, referring to FIG. 4, when there are six magnetic force generating units, the angles formed by at least two magnetic force generating units among the angles (θ1, θ2, θ3, θ4, θ5, θ6) formed by each magnetic force generating unit with respect to the rotation axis may be arranged to be different from each other. That is, one or more angles among the angles formed by the plurality of magnetic force generating units about the rotation axis may be different from other angles. Through this, the area of the magnetic field with uniform intensity formed on the reference plane may be further expanded.

In another example, the difference between the maximum value and the minimum value of the respective angles formed by the plurality of magnetic force generating units with respect to the rotation axis may be 5° or less. Referring to FIG. 4, it may refer to the difference between the angle having the maximum size and the angle having the minimum size among the angles (θ1, θ2, θ3, θ4, θ5, θ6) formed by the respective magnetic force generating units about the rotation axis. The difference between the angles may be 5° or less, 4° or less or 3° or less, and the lower limit is not particularly limited, but may be, for example, 0° or more. That is, angles formed by the plurality of magnetic force generating units with respect to the rotation axis may be substantially the same. In this case, the magnetic force generating unit may be rotated at a high speed by reducing the difference in centrifugal forces generated by the rotation of the plurality of magnetic force generating units.

### Bioactive Material

The system for modulating the activity of cells according to the present invention may modulate the activity of cells by applying a rotating magnetic field to be described below to generate a torque in the magnetic particles, and transmitting the torque generated to a bioactive material bound to the magnetic particles. That is, the bioactive material according to the present invention may have a “mechanosensitive” property.

In one example of the present invention, the bioactive material may include a mechanosensitive channel or a mechanosensitive ion channel. As used herein, the term “channel” may mean a channel that can be simultaneously opened in two regions divided into an inside and an outside by a membrane or the like, and may mean a tissue that allows a predetermined solute to pass through when opened. The “mechanosensitive channel or mechanosensitive ion channel” may mean a channel that is opened or closed by mechanical stress, and the solute may be diffused by opening the channel, and through this, the activity of cells may be modulated by the solute.

The mechanosensitive channel or mechanosensitive ion channel may be at least one selected from a cation channel and an anion channel, and the cation channel and anion channel may be at least one selected from the group consisting of a calcium channel, a potassium channel, a sodium channel and a chloride channel.

In one example, the mechanosensitive channel or mechanosensitive ion channel may include at least one selected from the group consisting of Piezo1, Piezo2, TRPC1, TRPC3, TRPC6, TRPM4, TRPM7, TRPN1, TRPA1, TRPY1, TRPP1, TRPP2, TRPV1, 1679K-TRPV1, TRPV2, TRPV4, TREK, TRAAK, ASIC1,2,3, MEC-4/MEC-10, MscL, MscS, RGD, integrin and cadherin. Further, in one example, the mechanosensitive channel or mechanosensitive ion channel may be Piezo1, but is not limited thereto. For example, when the channel is Piezo1, the channel may be a cation channel, and the channel may be opened according to the application of a rotating magnetic field to function to diffuse cations.

The cell of the system for modulating the activity of cells according to the present invention may be used to encompass a single cell, a cell population, an organ, an organ explant, a tissue and a tissue explant. In addition, the cell may be used to encompass dorsal root ganglion explants, nerve cells and glial cells, but is not limited thereto.

### Magnetic Particles

In an exemplary embodiment of the present invention, the magnetic particles of the system for modulating the activity of cells according to the present invention may generate a torque of 10.0 pN·nm or more, when a rotating magnetic field satisfying the above-described relationship formulas is applied. The torque generated by the magnetic particles is transmitted to a bioactive material bound to the magnetic particles, and the bioactive material may be a channel having a mechanosensitive property as described above, and the channel may be opened or closed using the transmitted torque to modulate the activity of a desired cell. The upper limit of the torque generated by the magnetic particles when a rotating magnetic field satisfying Relationship Formulas 1, 2, 3 and/or 4 is applied is not particularly limited as long as it does not damage the bioactive material, but for example, it may be 1 μN·μm or less.

In one example, the average particle diameter of the magnetic particles may be 2.0 μm or less. In the present specification, the particle size of the magnetic particles may refer to the particle size of the magnetic particles to which nanoparticles are attached to the surface of the core (refer to FIG. 13). The average particle diameter of the magnetic particles is not particularly limited, but may mean, for example, a D50 particle diameter measured by dynamic light scattering. The average particle diameter of the magnetic particles may be 2.0 μm or less, 1.8 μm or less, 1.6 μm or less, 1.4 μm or less, 1.2 μm or less or 1.0 μm or less. In addition, the lower limit of the average particle diameter of the magnetic particles is not particularly limited, but may be, for example, 50 nm or more or 100 nm or more. FIG. 19 shows the relationship between the size of magnetic particles according to the present invention and the number of magnetic particles bound to cells. Referring to FIG. 19, it can be confirmed that as the size of the magnetic particles increases, the number of magnetic particles bound to one cell decreases. This is because there is a constraint on the space for the magnetic particles to bind to the small-sized cells, and when the size of the magnetic particles is 3 μm, substantially only one or two magnetic particles can be bound to one cell. If the average particle diameter of the magnetic particles exceeds the above range, the number of magnetic particles binding to the cells may be too small, and thus, a sufficient level of torque may not be generated. In addition, if the size of the magnetic particles is too small, it may be difficult to manufacture the magnetic particles having a desired shape or it may be difficult to generate a desired level of torque.

In an exemplary embodiment of the present invention, the magnetic particles according to the present invention may be magnetically anisotropic (magnetic anisotropy). In the present specification, magnetic anisotropy may mean a property of different magnetic permeability, which is the degree of magnetization depending on the direction of the magnetic field with respect to the crystal axis when a magnetic field is applied to the crystal of a magnetic material. In conventional magnetogenetics, a single particle is generally used as a magnetic particle. However, when a single particle is used, there is a limitation to the coercive force even if a shape such as a disk shape is used, and if the size of the magnetic particle using a magnetic material such as a metal is too large, the attached bioactive material is inevitably adversely affected. As the magnetic particles according to the present invention have a structure in which nanoparticles exhibiting magnetic anisotropy are disposed on the surface of the core as described below, a small amount of nanoparticles may be used, and it is possible to generate a large amount of torque.

In an exemplary embodiment of the present invention, the magnetic particles for modulating the activity of cells according to the present invention may include a core and a plurality of nanoparticles disposed on the surface of the core. As used herein, the term “core” may mean a region located inside a plurality of nanoparticles disposed outside the magnetic particles, and may mean a carrier having a function of supporting and/or immobilizing the plurality of nanoparticles. FIG. 13 schematically shows the magnetic particles of the present invention. Referring to FIG. 13, the magnetic particles according to the present invention may have a structure in which a core 11 is positioned therein, and a plurality of nanoparticles 12 are disposed to surround the core 11. In this case, the core 11 may have a spherical shape, but is not limited thereto. As used herein, the term “spherical” may mean a shape in which no angle exists on the outer surface, and may mean a three-dimensional figure in which cut surfaces in all directions are circular. In addition, the spherical/circular shape may include not only a perfect spherical/circular shape, but also a shape that is close to a spherical shape/elliptical shape observed from the outside. Since the magnetic particles according to the present invention have the above structure, it is possible to increase the sensitivity to the rotating magnetic field.

The core is not particularly limited as long as it can attach nanoparticles to the surface, but may include, for example, inorganic materials such as polymers, ceramics, metal matrix composites (MMC), ceramic matrix composite materials (CMC) or the like. Specific examples of the polymer may include at least one selected from the group consisting of polyamide, polyurethane, polyethylene, polypropylene, polyacetal, polycarbonate, polybutylene terephthalate (PBT), polyethylene terephthalate (PET), polyethylene naphthalate (PEN), polytrimethylene terephthalate (PTT), polyhexamethylene terephthalate, polystyrene, polyvinyl chloride, polytetrafluoroethene, polyester (e.g. polylactic acid), polyether, polyether sulfone, polyether ether ketone, polyacrylate, polymethacrylate, polyimide, acrylonitrile butadiene styrene (ABS), polyphenylene sulfide, vinyl polymer, polyarylene ether, polyarylene sulfide, polysulfone, polyether ketone, polyamide-imide, polyetherimide, polyetherester, copolymers including polyether blocks and polyamide blocks (PEBA or polyether block amide), grafted or ungrafted thermoplastic polyolefin, functionalized or non-functionalized ethylene/vinyl monomer polymer, functionalized or non-functionalized ethylene/alkyl (meth)acrylate, functionalized or non-functionalized (meth)acrylic acid polymer, functionalized or non-functionalized ethylene/vinyl monomer/alkyl (meth)acrylate terpolymer, ethylene/vinyl monomer/carbonyl terpolymer, ethylene/alkyl (meth)acrylate/carbonyl terpolymer, methylmethacrylate-butadiene-styrene (MBS)-type core-shell polymer, polystyrene-block-polybutadiene-block-poly(methyl methacrylate) (SBM) block terpolymer, chlorinated or chlorosulfonated polyethylene, polyvinylidene fluoride (PVDF), phenolic resin, poly(ethylene/vinyl acetate), polybutadiene, polyisoprene, styrene-based block copolymer, polyacrylonitrile, silicone, silica and combinations thereof, but the present invention is not limited thereto.

Specific examples of the ceramic may be exemplified by at least one selected from the group consisting of Si, Al, Mg, Zn, Ba, Co, Mn, Mo, Ni, W, Cr, Ti, Zr, B, Hf, Yb, Ce, Y, Bi, Er, Sm, Gd, Dy, V, oxides thereof, nitrides thereof and carbides thereof, but the present invention is not limited thereto. In addition, specific examples of the metal matrix composite material (MMC) and/or the ceramic matrix composite material (CMC) may be exemplified by composite matrices of the ceramic and a metal component that can be used as nanoparticles to be described below, but the present invention is not limited thereto.

A plurality of nanoparticles may be attached to the surface of the core. The plurality of nanoparticles may be magnetizable nanoparticles, and specifically, nanoparticles having magnetic sensitivity. As the system for modulating the activity of cells according to the present invention uses magnetic particles having a structure in which a plurality of magnetizable nanoparticles are attached to the surface of the core, a sufficient torque may be generated even when a low-strength rotating magnetic field is applied, and it is possible to effectively modulate the activity of cells.

In an exemplary embodiment of the present invention, the nanoparticles disposed on the surface of the magnetic particles may be magnetically anisotropic (magnetic anisotropy). As the magnetic particles according to the present invention have a structure in which nanoparticles exhibiting magnetic anisotropy are disposed on the surface of the core, a small amount of nanoparticles may be used, and it is possible to generate a large amount of torque.

The shape of the nanoparticles may be a spherical shape or a polyhedral shape. As used herein, the term “polyhedron” may mean a three-dimensional shape in which a plurality of polygons are combined, and may mean a three-dimensional structure in which a plurality of polygons are combined without a gap. In addition, the polygon may mean a shape formed by connecting a finite number of line segments, and may mean a structure having a vertex at a junction where at least two line segments meet. The nanoparticles of the magnetic particles according to the present invention may have, for example, an octahedral shape, but is not limited thereto. Since the nanoparticles have a polyhedral shape, it is possible to effectively exhibit magnetic anisotropy.

In an exemplary embodiment of the present invention, the average particle diameter of the nanoparticles of the magnetic particles included in the system for modulating the activity of cells of the present invention may be more than 5 nm. The average particle diameter of the nanoparticles is not particularly limited, but may mean a D50 particle diameter measured by dynamic light scattering. The average particle diameter of the nanoparticles may be more than 5 nm, 6 nm or more, 8 nm or more, 10 nm or more, 14 nm or more, 18 nm or more, 20 nm or more, 22 nm or more, 24 nm or more or 25 nm or more, and the upper limit is not particularly limited, but may be, for example, 100 nm or less. The nanoparticles must have magnetic anisotropy, and at the same time, the magnetic particles must be attached to the core in a sufficient amount to generate a high torque. If the nanoparticles are too small, it is difficult to have magnetic anisotropy, and if the nanoparticles are too large, a sufficient amount of nanoparticles may not be attached to one magnetic particle, or dispersibility in a solution may be very poor during the manufacturing process.

If the nanoparticles are a material having magnetic sensitivity, the composition thereof is not particularly limited. Non-limiting examples of components that can be used as the nanoparticles may be at least one selected from the group consisting of iron oxide, zinc ferrite, manganese (Mn), nickel (Ni), cobalt (Co), bismuth (Bi), platinum (Pt), gold (Au), palladium (Pd), copper (Cu), alloys thereof or ferrites thereof, but the present invention is not limited thereto. In addition, the iron oxide may be at least one selected from the group consisting of iron(II) oxide (FeO), iron(III) oxide (magnemite; Fe₂O₃, α-Fe₂O₃, β-Fe₂O₃, γ-Fe₂O₃, ε-Fe₂O₃), magnetite (Fe₃O₄), iron(II, III) oxides (Fe₄O₅, Fe₅O₆, Fe₅O₇, Fe₁₃O₁₉, Fe₂₅O₃₂) and alloys thereof, but the present invention is not limited thereto.

In one example, the magnetic particles according to the present invention may further include a linker which links the core and the nanoparticles. The core and the nanoparticles may be coupled to each other through a linker. The term “coupling” may mean that any one material is bonded to another material, and the bonding may mean that it includes both a covalent bond and a non-covalent bond. For example, the non-covalent bond may include charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions and/or combinations thereof, but is not limited thereto.

The linker may include at least one selected from the group consisting of amide linkers, disulfide linkers, triazole linkers, thioether linkers, hydrazone linkers, hydrazide linkers, imine or oxime linkers, urea or thiourea linkers, amidine linkers, amine linkers and sulfonamide linkers.

In one example, the magnetic particles according to the present invention may include one or more moieties for binding to the antigen or specific receptor of a bioactive material. As used herein, the term “antigen” may refer to a molecule that induces an immune response, and the immune response may include antibody production and/or activation of specific immunologically suitable cells. The antigen may be derived from an organism, a subunit of a protein/antigen, a killed or inactivated whole cell or a lysate. When the bioactive material includes an antigen, the magnetic particles may include an antibody as the moiety. As used herein, the term “antibody” may refer to a specific protein molecule directed against an antigenic site, and the term “specific binding” refers to a specific sample, for example, a nucleic acid molecule, polypeptide, or complex thereof (e.g., a binding protein, such as a transcription factor and a homologous nucleic acid binding region thereof), or compound or molecule that recognizes a given polypeptide and/or nucleic acid molecule in a biological sample and is capable of binding thereto, and at the same time substantially recognizes and not binding to another molecule. The magnetic particles of the system for modulating the activity of cells according to the present invention include one or more moieties for binding to the antigen or specific receptor of a bioactive material such that even when injected into the living body, the effect on other cells is minimized and only the activity of the desired cell may be selectively adjusted.

In addition, the present invention relates to a method for modulating the activity of cells. The method for modulating the activity of cells according to the present invention may include a magnetic field application step of applying a rotating magnetic field which satisfies Relationship Formulas 1 and 2 below to magnetic particles capable of binding to a bioactive material and generating a torque when a rotating magnetic field is applied to transmit the torque to the bioactive material; and a torque transmission step of transmitting the torque generated according to the application of the rotating magnetic field to the bioactive material:

$$\vert {M}_{c} \vert \geq 1 mT \qquad [ Relationship Formula 1 ]$$

$$\vert {M}_{75} - {M}_{c} \vert / {D}_{75} \leq 5. T / m \qquad [ Relationship Formula 2 ]$$

In the relationship formulas above, M_{c} is the strength of the magnetic field at the position of the rotation axis, D₇₅ is the distance from the rotation axis to the 75% position of the distance to the magnetic force generating unit, and M₇₅ is the strength of the magnetic field at the position D₇₅.

In addition, the method for modulating the activity of cells according to the present invention may apply a rotating magnetic field which satisfies Relationship Formula 3.

$$\vert {M}_{50} - {M}_{c} \vert / {D}_{50} \leq 1 T / m \qquad [ Relationship Formula 3 ]$$

In the relationship formula above, M_{c} is the strength of the magnetic field at the position of the rotation axis, D₅₀ is the distance from the rotation axis to the 50% position of the distance to the magnetic force generating unit, and M₅₀ is the strength of the magnetic field at the position D₅₀.

The method for modulating the activity of cells according to an exemplary embodiment of the present invention may apply a rotating magnetic field which satisfies Relationship Formula 4 below.

$$\vert {M}_{75} - {M}_{50} \vert / ( {D}_{75} - {D}_{50} ) \leq 10 T / m \qquad [ Relationship Formula 4 ]$$

In Relationship Formula 4 above, D₇₅ is the distance from the rotation axis to the 75% position of the distance to the magnetic force generating unit, D₅₀ is the distance from the rotation axis to the 50% position of the distance to the magnetic force generating unit, M₇₅ is the strength of the magnetic field at the position D₇₅, and M₅₀ is the strength of the magnetic field at the position D₅₀.

Since the descriptions of the configuration and operation of the rotating magnetic field applying device are the same as those described above, they will be omitted.

The bioactive material in the rotating magnetic field application step may include a mechanosensitive channel and/or a mechanosensitive ion channel. Since the descriptions of the channel and the mechanosensitive channel or mechanosensitive ion channel is the same as those described above, they will be omitted.

The magnetic particles of the method for modulating the activity of cells according to the present invention may generate a torque of 10 pN·nm or more when a rotating magnetic field satisfying at least one of Relationship Formulas 2 to 4 is applied while satisfying Relationship Formula 1 above.

In an exemplary embodiment of the present invention, the area of the rotating magnetic field satisfying at least one of Relationship Formula 2 to 4 while satisfying aforementioned Relationship Formula 1 may be 0.1 cm² or more. The area of the rotating magnetic field may be 0.1 cm² or more. 0.2 cm² or more, 0.3 cm² or more, 0.4 cm² or more, 0.5 cm² or more, 0.6 cm² or more, 0.7 cm² or more, 0.8 cm² or more, 0.9 cm² or more or 1.0 cm² or more, but is not limited thereto. In addition, the upper limit of the area of the rotating magnetic field satisfying the above relationship formulas is not particularly limited, but may be, for example, 10 m² or less.

The method for modulating the activity of cells according to the present invention may include a torque transmission step of transmitting a torque generated according to the application of a rotating magnetic field to a bioactive material bound to magnetic particles. The torque transmission step may be a step in which a torque generated from magnetic particles according to the application of a rotating magnetic field is transmitted through a surface of the bioactive material to which the magnetic particles are bound. In this case, the magnetic particles may be attached to the surface of the bioactive material.

In this case, the mechanosensitive channel and/or the mechanosensitive ion channel of the method for modulating the activity of cells according to the present invention may be opened or closed according to the application of a rotating magnetic field. The method for modulating the activity of cells according to the present invention may open or close the mechanosensitive channel and/or the mechanosensitive ion channel by applying a rotating magnetic field.

Meanwhile, according to an exemplary embodiment of the present invention, the method for modulating the activity of cells of the present invention may further include a magnetic particle attachment step performed before the magnetic field application step. The magnetic particle attachment step may be a step of binding the magnetic particle to a specific receptor or antigen on the bioactive material. The magnetic particle attachment step may be performed in vivo or ex vivo. For example, the magnetic particle attachment step may be performed in such a way that the antibody attached to the surface of the magnetic particle and the antigen expressed on the bioactive material bind, but is not limited thereto. By allowing the magnetic particles to bind to a specific receptor or antigen on the bioactive material as described above, the magnetic particles may be bound to the desired bioactive material, and at the same time, binding of the magnetic particles to a region other than the desired bioactive material is inhibited, and thus, it is possible to selectively modulate the activity of the target cell.

Further, in an exemplary embodiment of the present invention, the method for modulating the activity of cells according to the present invention may further include a step of activating cells using the torque transmitted to the bioactive material. As described above, the method for modulating the activity of cells according to the present invention receives the torque generated by the application of a rotating magnetic field and opens the mechanosensitive channel and/or the mechanosensitive ion channel such that the solute may be diffused through the channel, and for example, cells that respond to the solute may be activated or deactivated. Alternatively, the concentration of the solute, which was maintained constant through the open mechanosensitive channel and/or mechanosensitive ion channel, may be adjusted such that the concentration of the solute is different in each region in both directions of the channel by closing the channel.

In this case, the cells activated in the step of activating the cells may include nerve cells, glial cells, immune cells and/or cancer cells.

Hereinafter, the present invention will be described in more detail through the examples and comparative examples. However, the spirit of the present invention is not limited to the examples to be described below.

### Magnetic Particles

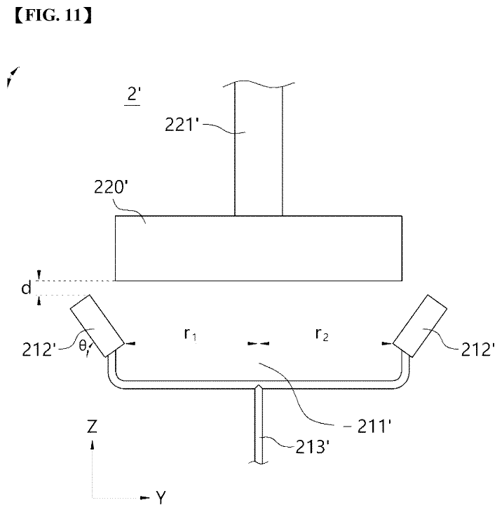

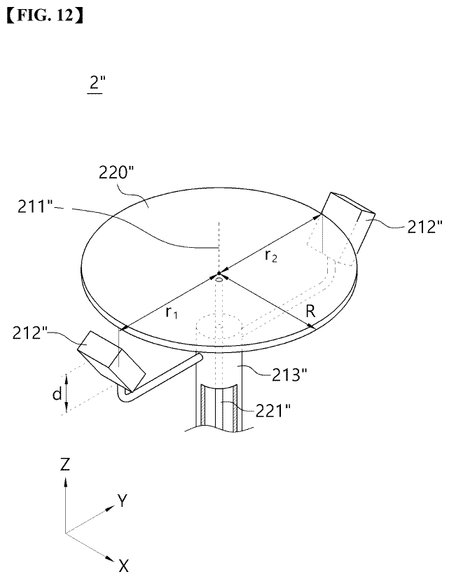

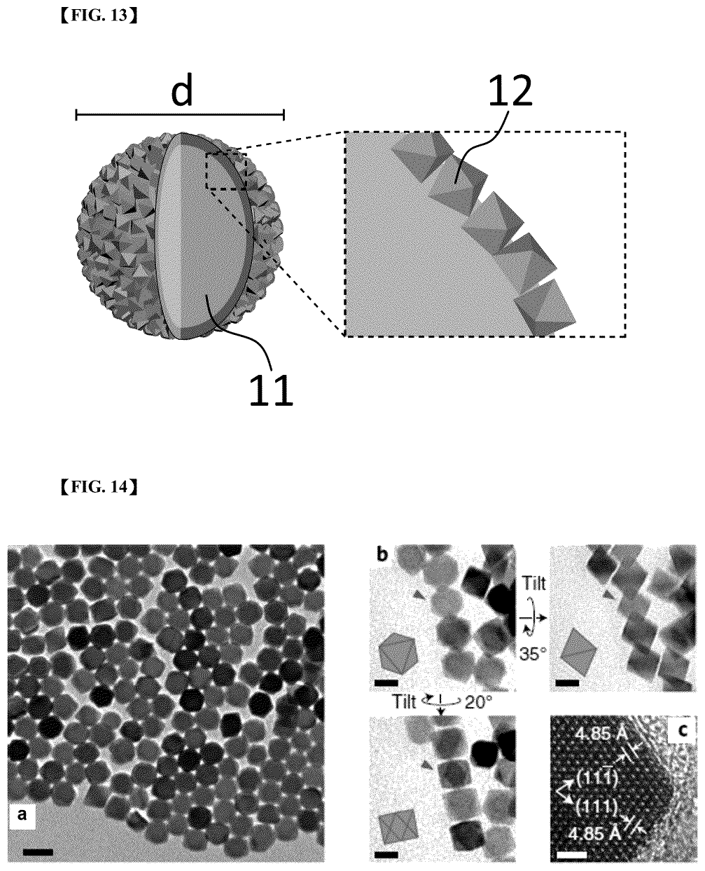

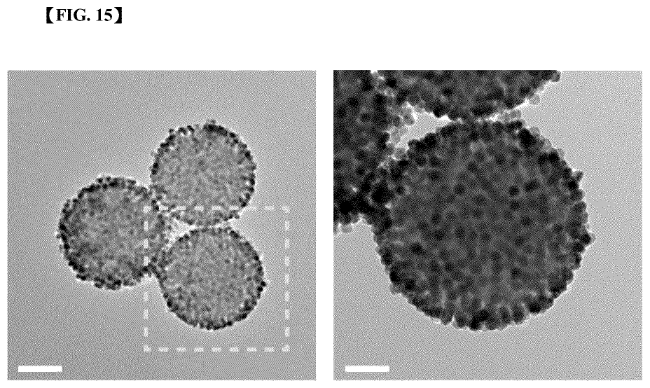

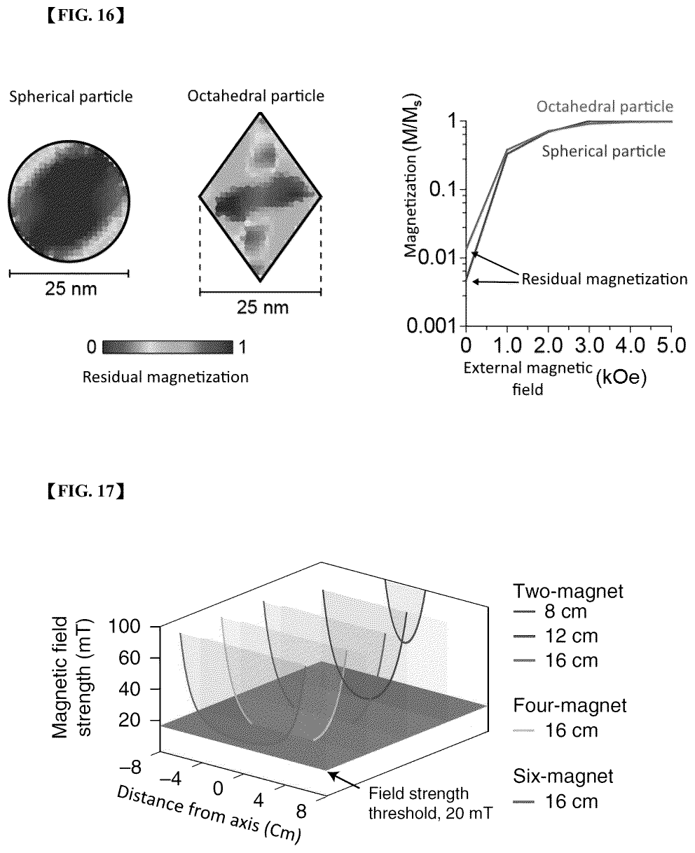

Magnetic particles were prepared in the following way. Spherical polystyrene having an average particle diameter of 500 nm was used as a core (manufactured by Polysciences), and iron oxide (Fe₃O₄) having an average particle diameter of 25 nm and an inverted spinel structure having an octahedral shape was prepared and used as nanoparticles. FIG. 14 is a set of TEM images obtained by photographing nanoparticles used for synthesizing the magnetic particles. Referring to FIG. 14, it can be confirmed that the nanoparticles had an octahedral shape having a particle diameter of about 25 nm. FIG. 16 is a simulation result of the residual magnetization of nanoparticles at room temperature using a micromagnetism simulation program (OOMMF). Referring to FIG. 16, it can be confirmed that the nanoparticles had magnetic anisotropy.

1 mg of the nanoparticles was dispersed in dimethyl sulfoxide (DMSO), and 0.1 mg of 1,2,3-triazole azido-dPEG24-TFP ester (manufactured by Quantabiodesign) was added to bind the nanoparticles and azide. Afterwards, 0.5 mg of the polystyrene core was dispersed in DMSO, and 1 mg of DBCO-PEG4-NHS (manufactured by Click Chemistry Tools) was added to bind DBCO to the polystyrene core. Each particle was separated by centrifugation and stirred at 25° C. for 8 hours to form 1,2,3-triazole, thereby preparing magnetic particles having nanoparticles attached to the surface of the polystyrene core. FIG. 15 is a set of TEM images of the prepared magnetic particles. Referring to FIG. 15, it can be confirmed that the iron oxide nanoparticles were attached to the surface of the polystyrene core.

### Rotating Magnetic Field Generating Device

FIG. 1 is a mimetic diagram of the rotating magnetic field generating device according to the present invention. The rotating magnetic field generating device was manufactured to have a different number of magnets and a different interval between the long axes. The rotating magnetic field generating device was manufactured such that the space in the center does not rotate even when the external magnet rotates. The strengths of the magnetic field measured by varying the number of magnets and the distance between the major axes in the manufactured rotating magnetic field generating device are shown in Table 1 below.

TABLE 1

| Classification | | | | | | | | | |
|---|---|---|---|---|---|---|---|---|---|
| (Distance of | | | | | | | Change rate of magnetic | | |
| long axis/ | Strength of magnetic | | | Change in magnetic | | | field (T/m) | | |
| number of | field (mT) | | | field (Δ) (mT) | | | \|M₇₅ − M_{c}\|/ | \|M₅₀ − M_{c}\|/ | \|M₇₅ − M₅₀\|/ |
| magnets) | \|M_{c}\| | \|M₅₀\| | \|M₇₅\| | \|M₇₅ − M_{c}\| | \|M₅₀ − M_{c}\| | \|M₇₅ − M₅₀\| | Δx | Δx | Δx |
| 8 cm/ | 53 | 102 | 206 | 153 | 49 | 104 | 5.1 | 2.45 | 10.4 |
| 2 magnets | | | | | | | | | |
| 12 cm/ | 21 | 50 | 136 | 115 | 29 | 86 | 2.56 | 0.97 | 5.73 |
| 2 magnets | | | | | | | | | |
| 16 cm/ | 13 | 33 | 104 | 91 | 20 | 71 | 1.52 | 0.5 | 3.55 |
| 2 magnets | | | | | | | | | |
| 16 cm/ | 15 | 29 | 92 | 77 | 14 | 63 | 1.28 | 0.35 | 3.15 |
| 4 magnets | | | | | | | | | |
| 16 cm/ | 25 | 36 | 88 | 63 | 11 | 52 | 1.05 | 0.275 | 2.6 |
| 6 magnets | | | | | | | | | |
| 70 cm/ | 26 | 45 | 100 | 74 | 19 | 55 | 0.282 | 0.109 | 0.63 |
| 10 magnets | | | | | | | | | |

Referring to Table 1, when the lengths of the long axes were 12 cm, 16 cm and 70 cm, respectively, it can be confirmed that all of Relationship Formula 1 to 4 were satisfied even if only two magnets were disposed. However, when the length of the long axis was 8 cm and two magnets were used, it can be seen that the strength of the magnetic field changed rapidly as it moved away from the position of the rotation axis. In particular, it showed a very large change rate of the magnetic field with respect to Relationship Formula 3.

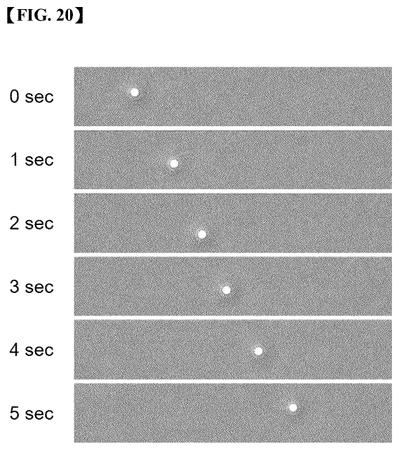

FIG. 20 is the results of placing magnetic particles in the rotating magnetic field generating device when the length of the long axis was 8 cm and two magnets were used. Referring to FIG. 20, when a magnetic field having a greater change rate of magnetic field was applied compared to each of Relationship Formulas 2, 3 and 4, it can be confirmed the physical position itself changed along the direction of magnetic flux, rather than causing the magnetic particles to rotate to generate torque. This may mean that when the rotating magnetic field does not satisfy any one or more of Relationship Formulas 2 to 4, the magnetic particles do not generate torque. In this case, it is not possible to transmit a torque at a required degree to the bioactive material, and only a weak linear magnetic force is applied such that it may not be possible to modulate the activity of the cell. In addition, when the force that changes the position along the magnetic flux direction is strong, it may damage the bioactive material to which the magnetic particles are bound, and further may cause irreversible damage to the cell itself.

### Example 1

In order to confirm the action of the magnetic particles and the rotating magnetic field generating device, a fluorescence material (fluorescence probe green, 100 nm, manufactured by Sigma-Aldrich) was attached to one of the surfaces of the aforementioned magnetic particles. After the magnetic particles to which the fluorescent material was attached were placed in the central region of the rotating magnetic field generating device, a magnetic field of 25 mT was applied and the external magnet was rotated at a speed of 0.5 Hz.

### Comparative Example 1

The same magnetic particles as in Example 1 were used except that a linear magnetic field generating device was used, and it was observed after applying a magnetic field at the same strength (25 mT).

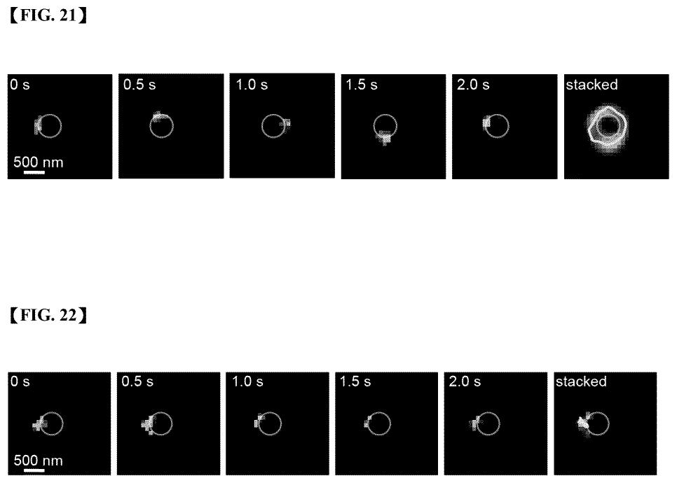

FIG. 21 is a set of images of the magnetic particles according to Example 1 of the present invention photographed with a fluorescence microscope, and FIG. 22 is a set of images of the magnetic particles according to Comparative Example 1 photographed with a fluorescence microscope. In FIGS. 21 and 22, white circles in the center indicate an outline of the magnetic particle. Referring to FIG. 21, according to Example 1 of the present invention, it can be confirmed that the magnetic particles rotated with time. In addition, it can be confirmed that it took about 2 seconds for the magnetic particles to rotate once (360°). This indicates that the magnetic particles may be rotated at a desired speed in the configuration according to Example 1 of the present invention, and thus, it can be seen that torque may be transmitted.

On the other hand, referring to FIG. 22, it can be confirmed that the magnetic particles did not move according to Comparative Example 1 of the present invention. This means that the position of magnetic particles cannot be changed even if a linear magnetic field at the same strength as the rotating magnetic field is applied, and even if the same magnetic particles are moved, it may mean that a linear magnetic field with greater strength than the rotating magnetic field is required. In addition, it may mean that a sufficient torque cannot be generated only by a linear magnetic field rather than a rotating magnetic field.

### Example 2

In order to confirm the activity in neurons, the pENTCMV-Myc897-Piezo1 gene capable of expressing the Piezo1 channel was injected into adenovirus, which was then infected in the cortical neurons of mice. FIG. 23 is a set of immunostaining fluorescence images obtained by photographing a fluorescent material stained with the Myc antibody in the Piezo1 channel. Referring to FIG. 23, it can be confirmed that the Piezo1 channel was expressed in a sufficient amount in the cortical neurons of mice by the injected adenovirus.

Afterwards, in order to attach the magnetic particles to the Piezo1 ion channel, the Myc antibody was attached to the surface of the magnetic particles described above by using EDC/NHS chemistry and Protein A. Afterwards, a saline solution containing magnetic particles was injected, and sufficient time was allowed such that it was sufficiently introduced into the cortical neurons of the mice.

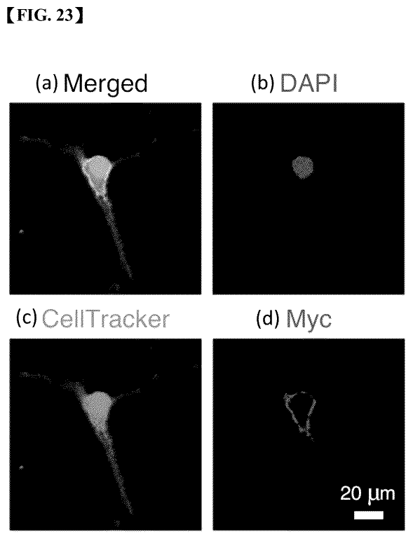

FIG. 24 is a set of images obtained by photographing the distribution of magnetic particles in cortical neurons of the rat with a fluorescence microscope. Referring to FIG. 24, it can be confirmed that the fluorescence signal of the magnetic particles was detected in the neurons expressing Piezo1. Through this, it can be confirmed that the injected magnetic particles were arranged by targeting the Piezo1 channel.

### Comparative Example 2

Magnetic particles were injected into neurons and a rotating magnetic field was applied to the neurons under the same conditions as in Example 2, except that the magnetic particles were not bound to the Piezo1 channel but were bound to WGA, which non-specifically binds to the cell surface.

### Comparative Example 3

Magnetic particles were bound to the Piezo1 channel and a rotating magnetic field was applied in the same manner as in Example 2, except that Ruthenium Red for inactivating the Piezo1 channel was additionally injected.

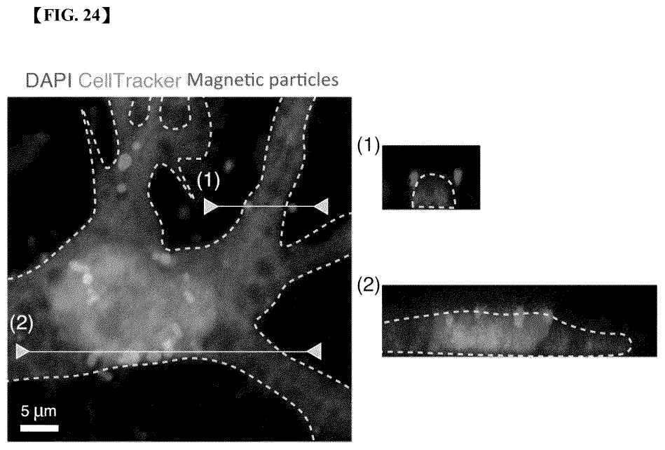

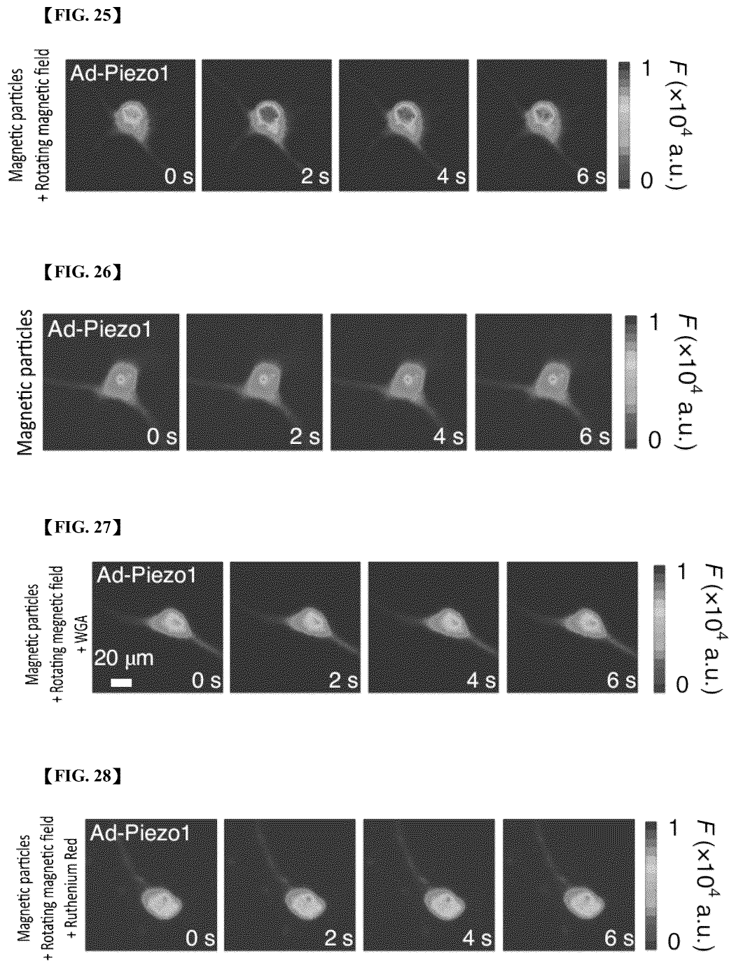

FIG. 25 is a set of images obtained by photographing Ca²⁺ ion-sensitized fluorescence signals (X-Rhod-1) when a rotating magnetic field was applied at a rate of 0.5 Hz after placing the neurons of Example 2 in the above-described rotating magnetic field generating device, and FIG. 26 is a set of images obtained by photographing Ca²⁺ ion-sensitized fluorescence signals (X-Rhod-1) when no rotating magnetic field was applied to the neurons of Example 2 above. Regions marked in red in FIGS. 25 and 26 indicate regions in which Ca²⁺ ions were activated. Referring to FIG. 25, it can be confirmed that Ca²⁺ ions were released, when the rotating magnetic field according to the present invention was applied. In addition, referring to FIG. 26, even when the magnetic particles according to the present invention were injected into neurons, it can be confirmed that Ca²⁺ ions were not activated if a rotating magnetic field was not applied. Through this, it can be confirmed that Ca²⁺ ions were released only when a rotating magnetic field was applied to the neuron to which the magnetic particles were attached, rather than the magnetic particles were attached to the neurons to release Ca²⁺ ions.

On the other hand, in the case of Comparative Example 2, there was no change in the Ca²⁺ activity. FIG. 27 is a set of images obtained by photographing the Ca²⁺ ion-sensitized fluorescence signals of Comparative Example 2. Referring to FIG. 27, it can be seen that Ca²⁺ ions were not activated even when the same rotating magnetic field was applied when the magnetic particles were not coupled to the Piezo1 channel.

Further, in Comparative Example 3, there was no change in the Ca²⁺ activity. FIG. 28 is a set of images obtained by photographing the Ca²⁺ ion-sensitized fluorescence signals of Comparative Example 3. Referring to FIG. 28, it can be seen that even if the magnetic particles were coupled to the Piezo1 channel, Ca²⁺ ions were not activated even when the same rotating magnetic field was applied when the Piezo1 channel was inactivated by injecting Ruthenium Red.

When a predetermined rotating magnetic field was applied to the magnetic particles according to Example 1 described above, it can be confirmed that the magnetic particles rotated. Further, in the case of Example 2, it can be seen that when a predetermined rotating magnetic field was applied, the Piezo1 channel was activated. Further, in the case of Example 2 in which the magnetic particles were coupled to Piezo1, it can be confirmed that Ca²⁺ ions were released by rotation of the magnetic particles. In addition, through Comparative Example 3, it can be confirmed that the Piezo1 channel functioned as a Ca²⁺ ion channel.

Therefore, in the case of Example 2 in which Piezo1 and magnetic particles were coupled, magnetic particles rotated according to the application of a rotating magnetic field to generate torque, and the torque was transmitted to the Piezo1 channel coupled with the magnetic particles, and as the Piezo1 channel opened, it can be confirmed that Ca²⁺ ions were released.

### Example 3 and Comparative Examples 4 and 5

For the same magnetic particles as those used in Example 1, the force generated by changing the strength of the magnetic field was measured. The force was measured by observing the maximum speed (ωₘₐₓ) of particle rotation through a fluorescence microscope (τ=8πηr³ωₘₐₓ). For comparison, Comparative Example 4 (ThermoFisher, Dynabead MyOne) and Comparative Example 5 (Spherotech, Carboxyl superparamagnetic particles) using nanoparticles exhibiting magnetic isotropy were used in the same manner to measure the change in the forces according to the strength of the magnetic field. The magnetic particles used in Comparative Example 4 had a structure in which nanoparticles (iron oxide) were uniformly dispersed inside spherical polystyrene, and the magnetic particles used in Comparative Example 5 had a structure in which iron oxide layers and polystyrene layers were alternately stacked on a spherical polystyrene core.

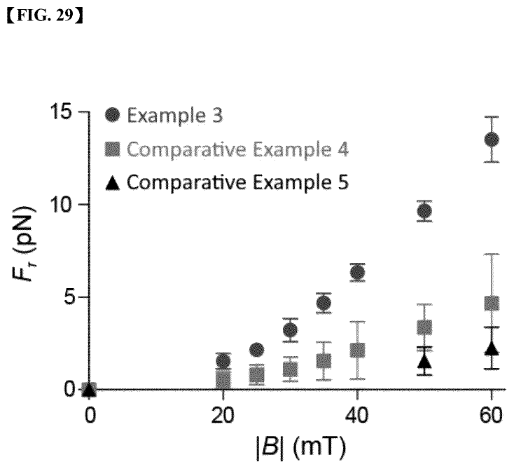

FIG. 29 shows the results of Example 3 and Comparative Examples 4 and 5. Referring to FIG. 29, it can be confirmed that, unlike Comparative Examples 4 and 5 using nanoparticles exhibiting magnetic isotropy, the magnetic particles of the present invention can transmit a very large force even when the same amount is used.

In particular, it can be seen that Comparative Example 4, which had a structure in which a plurality of iron oxide particles were dispersed in a spherical polystyrene bead, generated a lower level of force compared to Example 3 even though the structure included a plurality of iron oxide nanoparticles. Further, in the case of Comparative Example 5, which had an iron oxide layer surrounding the spherical core, it can be confirmed that only a very low level of torque was generated compared to Example 3 even though the iron oxide shell was disposed outside the core.

Although the exemplary embodiment of the present invention has been described in detail above, the present invention is not limited by the above-described exemplary embodiment and the accompanying drawings, but is intended to be limited by the appended claims. Accordingly, various types of substitution, modification and change will be possible by those skilled in the art within the scope not departing from the technical spirit of the present invention described in the claims, and it is also said that these fall within the scope of the present invention.

### EXPLANATION OF REFERENCE NUMERALS

11: Core
12: Nanoparticles
111: Rotation axis
112: Magnetic force generating unit
113: Driving unit
120: Reference plane
121: Support

1. A method of modulating the activity of target cells or of a living body comprising the target cells, the method comprising: (a) attaching magnetic particles that generate a torque upon application of a rotating magnetic field to a bioactive material associated with the target cells; (b) positioning the target cells, a subject or at least a portion of a living body configured to be received within an internal space of a rotating magnetic field generating device that forms, about an imaginary rotation axis, a rotating magnetic field whose magnetic-flux direction rotates in a plane substantially perpendicular to the imaginary rotation axis; and (c) applying the rotating magnetic field so as to generate a torque in the magnetic particles and transmit the torque to the bioactive material, thereby modulating the activity of the target cells.

2. The method of claim 1, wherein, on a reference plane perpendicular to the imaginary rotation axis, the rotating magnetic field satisfies |M_{c}|≥1 mT and |M₇₅−M_{c}|/D₇₅≤5.0 T·m⁻¹, where M_{c} is a magnetic-field strength at the position of the rotation axis, D₇₅ is a distance from the rotation axis to 75% of a distance to a magnetic field generating unit, and M₇₅ is a magnetic-field strength at D₇₅.

3. The method of claim 2, wherein the rotating magnetic field further satisfies |M₅₀−M_{c}|/D₅₀≤1 T·m⁻¹, where D₅₀ is a distance from the rotation axis to 50% of the distance to the magnetic field generating unit, and M₅₀ is a magnetic-field strength at D₅₀.

4. The method of claim 3, wherein the rotating magnetic field further satisfies |M₇₅−M₅₀|/(D₇₅−D₅₀)≤10 T·m⁻¹.

5. The method of claim 2, wherein an area in which the rotating magnetic field satisfies at least the relationships of claim 2 is 0.1 cm² or greater.

6. The method of claim 2, wherein in at least a portion of the internal space the magnetic-field strength is at least 20 mT during operation.

7. The method of claim 6, wherein the rotating magnetic field is applied at about 0.5 Hz.

8. The method of claim 1, wherein the rotating magnetic field is generated by mechanically rotating permanent magnets or by electrically rotating a fixed set of coil-based electromagnets by phase control in a multi-axis arrangement.

9. The method of claim 1, wherein the magnetic particles generate a torque of at least 10 pN·nm when the rotating magnetic field satisfying at least the following relationship formulas is applied:
M_{c}|≥1 mT and |M₇₅−M_{c}|/D₇₅≤5.0 T·m⁻¹, where M_{c} is a magnetic-field strength at the position of the rotation axis, D₇₅ is a distance from the rotation axis to 75% of a distance to a magnetic field generating unit, and M₇₅ is a magnetic-field strength at D₇₅.

10. The method of claim 9, wherein the magnetic particles are magnetically anisotropic and have an average particle diameter of 2.0 μm or less.

11. The method of claim 10, wherein each magnetic particle comprises a core and a plurality of nanoparticles disposed on a surface of the core.

12. The method of claim 11, wherein the nanoparticles have an average particle diameter greater than 5 nm.

13. The method of claim 12, wherein the nanoparticles comprise at least one selected from Fe, Mn, Ni, Zn, Al, Co, Cr, Mo, Ti, Bi, Nd, Pt, Au, Pd, Cu, an alloy thereof, an oxide thereof, a ferrite thereof, or a doped ferrite thereof.

14. The method of claim 11, further comprising a linker that links the core and the nanoparticles.

15. The method of claim 1, wherein the bioactive material comprises a mechanosensitive channel or mechanosensitive ion channel and application of the rotating magnetic field opens or closes the channel.

16. A system for modulating the activity of target cells, comprising: (a) a rotating magnetic field generating device including an internal space sized to receive at least a portion of a living body; (b) a plurality of magnetic field generating units arranged around an imaginary rotation axis and operable to produce, on a reference plane perpendicular to the imaginary rotation axis, a rotating magnetic field whose magnetic-flux direction rotates about said axis; and (c) a driving unit coupled to rotate the plurality of magnetic field generating units or to electrically rotate the magnetic-flux direction; wherein, on said reference plane, the rotating magnetic field satisfies |M_{c}|≥1 mT and |M₇₅−M_{c}|/D₇₅≤5.0 T·m⁻¹.

17. The system of claim 16, wherein the device is configured to maintain, within an operating zone of the internal space, a magnetic-field strength of at least 20 mT, and wherein the rotating magnetic field further satisfies at least one of |M₅₀−M_{c}|/D₅₀≤1 T·m⁻¹ or |M₇₅−M₅₀|/(D₇₅−D₅₀)≤10 T·m⁻¹.

18. A rotating magnetic field generating device comprising: (a) an internal space sized to receive at least a portion of a living body; (b) a plurality of magnetic field generating units disposed around an imaginary rotation axis; and (c) a driving unit configured to rotate the plurality of magnetic field generating units or electrically rotate the magnetic-flux direction; wherein the device is configured to produce, on a reference plane perpendicular to the imaginary rotation axis, a rotating magnetic field having a substantially uniform field magnitude and a reduced field gradient within an operating zone of the internal space.

19. The device of claim 18, wherein the device is configured to produce, on a reference plane perpendicular to the imaginary rotation axis, a rotating magnetic field satisfying |M_{c}|≥1 mT and |M₇₅−M_{c}|/D₇₅≤5.0 T·m⁻¹, and wherein an area in which the rotating magnetic field satisfies |M_{c}|≥1 mT and at least one of |M₅₀−M_{c}|/D₅₀≤1 T·m⁻¹ or |M₇₅−M₅₀|/(D₇₅−D₅₀)≤10 T·m⁻¹ is 0.1 cm² or greater.

20. The device of claim 18, wherein the device is configured, during operation, to maintain within an operating zone a magnetic-field strength of at least 20 mT and a field-gradient magnitude of ≤10 T·m⁻¹, the rotating magnetic field being produced by mechanically rotating permanent magnets or by phase-controlled electromagnets.
